SEQUENCE LISTING (26 sequences)

SEQ ID NO: 1 - DNA, 21 bp, Artificial Sequence
  TPM3 forward primer
gagaggtatgaaggttattga

SEQ ID NO: 2 - DNA, 21 bp, Artificial Sequence
  TPM3 reverse primer
atcaccaacttacgagccacc

SEQ ID NO: 3 - DNA, 18 bp, Artificial Sequence
  RPS27 forward primer
ctttccggcggtgacgac

SEQ ID NO: 4 - DNA, 23 bp, Artificial Sequence
  RPS27 reverse primer
ttttatagcatcctgggcatttc

SEQ ID NO: 5 - DNA, 23 bp, Artificial Sequence
  HAX1 forward primer
tgagggccggacagagactacag

SEQ ID NO: 6 - DNA, 21 bp, Artificial Sequence
  HAX1 reverse primer
gtgggcaatgggtgagaggtg

SEQ ID NO: 7 - DNA, 23 bp, Artificial Sequence
  PYGO2 forward primer
agggccctgcatactcacatctg

SEQ ID NO: 8 - DNA, 18 bp, Artificial Sequence
  PYG)2 reverse primer
ccccctgcacacggaagc

SEQ ID NO: 9 - DNA, 22 bp, Artificial Sequence
  CKS1B forward primer
cttggcgttcagcagagtcagg

SEQ ID NO: 10 - DNA, 19 bp, Artificial Sequence
  CKS1B reverse primer
ggcgccggaacagcaagat

SEQ ID NO: 11 - DNA, 20 bp, Artificial Sequence
  ADAM15 forward primer
tccagccccagccaagacct

SEQ ID NO: 12 - DNA, 23 bp, Artificial Sequence
  ADAM15 reverse primer
gctcgcccggctccacaaacata

SEQ ID NO: 13 - DNA, 22 bp, Artificial Sequence
  CCT3 forward primer
gggtgcggtgattggcgactac

SEQ ID NO: 14 - DNA, 20 bp, Artificial Sequence
  CCT3 reverse primer
tgggggaaccggcagaacct

SEQ ID NO: 15 - DNA, 22 bp, Artificial Sequence
  PRCC forward primer
tgcctccgccccctcagatgct

SEQ ID NO: 16 - DNA, 22 bp, Artificial Sequence
  PRCC reverse primer
ctccccaactcccgccgcttca

SEQ ID NO: 17 - DNA, 24 bp, Artificial Sequence
  KIF14 forward primer
ctgctctacggctcacactaatgg

SEQ ID NO: 18 - DNA, 21 bp, Artificial Sequence
  KIF14 reverse primer
ctggcagcgggactaatcgta

SEQ ID NO: 19 - DNA, 22 bp, Artificial Sequence
  ELF3 forward primer
ctcgcctccccaccctcctctt

SEQ ID NO: 20 - DNA, 24 bp, Artificial Sequence
  ELF3 reverse primer
gcccctgctctgtcctctccatca

SEQ ID NO: 21 - DNA, 24 bp, Artificial Sequence
  TGFB@ forward primer
aatgccatcccgcccactttctac

SEQ ID NO: 22 - DNA, 24 bp, Artificial Sequence
  TGFB2 reverse primer
gccattcgccttctgctcttgttt

SEQ ID NO: 23 - DNA, 21 bp, Artificial Sequence
  AKT3 forward primer
gagcccaccattgttcatttg

SEQ ID NO: 24 - DNA, 18 bp, Artificial Sequence
  AKT3 reverse primer
gcacgccaccacccttcc

SEQ ID NO: 25 - DNA, 21 bp, Artificial Sequence
  GAPDH forward primer
gcggggctctccagaacatca

SEQ ID NO: 26 - DNA, 21 bp, Artificial Sequence
  GAPDH reverse primer
ccagccccagcgtcaaaggtg

(19) United States
(12) Patent Application Publication
(10) Pub. No.: US 2009/0181394 A1
(43) Pub. Date: Jul. 16, 2009

(54) Diagnostic methods and kits for hepatocellular carcinoma using comparative genomic hybridization

(75) Inventor: Yeun-Jun Chung, Seoul (KR)

Correspondence Address: CLARK & ELBING LLP, 101 FEDERAL STREET, BOSTON, MA, 02110, US

(73) Assignee: Catholic University Industry Academy Cooperation Foundation, Seoul (KR)

(21) Appl. No.: 12/319,929

(22) Filed: Jan. 14, 2009

(30) Foreign Application Priority Data
Jan. 15, 2008 (KR) 10-2008-0004627

(51) Int. Cl. C12Q 1/68; C40B 60/12

(52) U.S. Cl. 435/6; 506/39

(57) ABSTRACT

The invention provides diagnostic methods for determining the prognosis of hepatocellular carcinoma (HCC) comprising the steps of (a) observing recurrently altered genomic region on a chromosome; (b) measuring variation of one or more of RAR expression variations selected from the RAR variation group consists of gains of RAR-G1 to RAR-G14 and losses of RAR-L1 to RAR-L18 as defined in table 1, a diagnostic kit, and genes for useful in diagnosis or prognosis of liver cancer.

### SUMMARY OF THE INVENTION

The invention provides a diagnostic method for determining the prognosis of hepatocellular carcinoma (HCC) comprising the steps of (a) observing recurrently altered genomic region on a chromosome; (b) measuring variation of one or more of RAR expression variations selected from the RAR variation group consists of gains of RAR-G1 to RAR-G14 and losses of RAR-L1 to RAR-L18 as defined in table 1, a diagnostic kit, and genes for useful in diagnosis or prognosis of liver cancer.

### RELATED APPLICATIONS/PATENTS & INCORPORATION BY REFERENCE

The present application claims the benefit of Korean Patent Application No. 10-2008-0004627 filed Jan. 15, 2008, the entire contents of which are hereby incorporated by reference.

Also, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

### FIELD OF THE INVENTION

The present invention is related to diagnostic methods and kits for hepatocellular carcinoma measuring expression of genes in recurrently altered genomic region (RAR) using comparative genomic hybridization.

### BACKGROUND OF THE INVENTION

Many genomic and genetic studies are directed to the identification of difference in gene dosage or expression among cell populations for the study and detection of disease. Many malignancies involve the gain or loss of DNA sequences that may result in activation of oncogene or inactivation of tumor suppressor genes.

Comparative genomic hybridization (CGH) is a technique that is used to evaluate variation s in genomic copy number in cells. In one implementation of CGH, genomic DNA is isolated from normal reference cells, as well as from test cells (e.g., tumor cells). The two nucleic acids are differentially labeled and then simultaneously hybridized to an array of oligonucleotide probes.

Array CGH assays measure the difference in copy number between a test sample and a reference sample. For example, two genomic samples (a test sample and a reference sample) can be labeled with two different dyes and hybridized together to a single microarray to perform these measurements. Alternatively, the two different samples can be hybridized to separate arrays and then measurement can be compared between arrays.

Hepatocellular carcinoma (HCC) is one of the most common human malignancies and responsible for approximately 5% of all cancer-related deaths in the world.¹ Given that the overall HCC incidence is still rising and complete resection of the lesion in early stage remains the only hope for cure, it is important to develop effective diagnostic and therapeutic modalities based on sound biological insights into hepatocarcinogenesis.^{2,3}

The copy number alterations observed in human solid tumors are known to contribute to the tumorigenesis by affecting the activities of cancer-related genes in the altered chromosomal regions.^{4,5} Thus, genome-wide mapping of copy number alterations in cancer can facilitate the identification of cancer-related genes, which will improve the understanding of tumorigenesis. Using conventional cytogenetic tools such as comparative genomic hybridization (CGH), copy number gains on 1q, 8q, and 20q, along with the losses on 1p, 4q, 8p, 13q, 16q, and 17p have been previously identified in HCC.⁶⁻⁸ However, the resolution of conventional cytogenetic analysis is insufficient to precisely identify sub-microscopic changes. Recently introduced array-CGH, combination of conventional CGH and microarray technology, enabled high-resolution screening of genome-wide copy number alterations containing potential cancer-related genes.^{5,9} Through array-CGH analysis, novel oncogenes such as JAB1 or differentiation-specific regions have been identified in HCC.^{6,10} But, considering the extensive and complex nature of chromosomal alterations, it is still difficult to identify biologically relevant changes and their functional significance in a systematic manner.

We hypothesized that recurrent copy number changes common to many HCC cases may contain essential genes for hepatocarcinogenesis. Using this strategy, recurrently altered regions (RAR) were defined in 76 primary HCCs using whole-genome array CGH analysis and the associations between RARs and clinicopathologic features were examined. Also, we functionally categorized the genes located in the RARs.

Korean patent registration No. 10-0534563 (registration date: Dec. 1, 2005) is related to a diagnostic method, composition, and primer for human hepatocellular carcinoma. Microarray and Representational Difference Analysis were performed to identify genes overexpressed in human hepatocellular carcinoma. The invention disclosed a diagnostic composition for human hepatocellular carcinoma comprising long chain fatty acid-coenzyme A ligase 4, farnesyl diphosphate synthase, syndecan-2, and a diagnostic method and primer using the composition.

Korean patent registration number 10-0552494 (registration date Feb. 8, 2006) disclosed genetic markers and diagnostic kits for liver cancer. More specifically, it disclosed useful diagnostic methods and diagnostic kits for diagnosis of liver cancer by comparing expression level of high expression gene and low expression gene.

Korean patent registration number 10-0527242 (registration date Nov. 2, 2006) is related to a microarray for measuring expression level of genes related to liver cancer. More specifically it is related to measurement and interpretation methods of molecular expression profile of protein and enzyme of liver cancer, which is characterized in that for measuring the profile of cDNA of protein and enzyme specifically expressed due to modified genes in hepatocellular carcinoma (HCC), a number of cDNAs obtained by reverse transcription of mRNAs from normal cell and liver cancer cell are mixed and bound with Cy5-dUTP probe, hybridized with 44 species of DNA microarray immobilized on DNA chip and then the result is analyzed by computer software.

As described above, there were a variety of diagnostic methods for liver cancer, however, there was no such a diagnostic method like present invention which determine the prognosis of a liver cancer by measuring recurrently altered genomic region (RAR) by using the method of comparative genomic hybridization,

### BRIEF DESCRIPTION OF THE DRAWINGS

FIG. 1. Whole-genome profiles and frequency plot of chromosomal alterations in hepatocellular carcinoma. (A) The genomic alterations in 76 primary human hepatocellular carcinomas are illustrated in individual lanes. A total of 2,958 large insert clones are mapped according to the UCSC genome browser (May, 2004 Freeze) and ordered by chromosomal position from 1pter to Yqter (X axis). Tumor versus reference intensity ratios (in log₂ scale) for individual clones are plotted in different color scales reflecting the extent of copy number gains (red) and losses (green) as indicated in the reference color bar. The boundaries of individual chromosome are indicated by long vertical bars and the locations of centromeres by short vertical bars below the plot. (B) The frequency of chromosomal gains (>3 SD) and losses (<−3 SD) for each clone is shown for 76 HCC samples.

FIG. 2. Expression levels of KIF14 and TPM3 by copy number status. Mean expression ratios (tumor/normal) of KIF14 (A) and TPM3 (B) were calculated by real-time qRT-PCR by presence of RAR. Human GAPDH gene was used as an internal control. Expression of KIF14 was significantly higher in samples with RAR-G2 (n=12) than without (n=8), and expression of TPM3 was significantly higher in samples with RAR-G1 (n=13) than without (n=7). X axis represents RAR status and Y axis represents mean expression ratio. Error bars represent 95% confidence intervals.

FIG. 3. Survival analysis of RARs in HCC. Kaplan-Meier survival curves. The survival curves for the cases with (blue line) or without (green line) specific genomic changes are plotted using the Kaplan-Meier method. The chromosomal changes associated with relatively poor survival are presented with the significance level. RAR-G, recurrently altered region of copy number gain; RAR-L, copy number loss

### DETAILED DESCRIPTION OF THE INVENTION

A confounding problem in genetic disease (especially cancer) diagnosis/prognosis has been the large amount of cellular heterogeneity in disease tissues. This is especially a problem for cancer tissues, partly due to their known tendency of chromosomal instability, and in some cases, different clonal origin and/or diverged progression from single clonal mutational events. Due to the nature of such disease tissues, there are no reliable methods to select only for tumor cell outgrowth for cytogenetic studies. This in turn has led to a high frequency of normal karyotypic findings for diseased tissues (false negative).

Genetic heterogeneity, which can be detected by conventional G-banding chromosome analysis, depends on the frequency of an aberrant clone and the number of cells analyzed, where the chromosomes of individual cells are analyzed. However, unlike the conventional cytogenetic approach of karyotype analysis, it is not the chromosomes of individual cells from a sample that are analyzed in microarray genome profiling, but rather the DNA sequence copy number of the total genomic DNA extracted from the cells of the sample. Consequently, from a DNA copy number perspective, the genome profile of a tumor maybe no different from that of total genomic DNA extracted from a reference population of 46, XX cells. Hence, the prior art has predicted that the genetic heterogeneity of this tumor sample would not be detected by microarray genome profiling.

The present invention is based at least in part that the detection of genetic heterogeneity in clinical samples, such that detection can be carried out under conditions and analysis to detect cell populations whose combined genetic profiles would have been predicted, e.g., by the prior art, to mask the presence of a heterogeneous population. In particular, the profiling methods of the present invention demonstrate the sensitivity with which it can detect clonally distinct cell populations within a more dominant background cell population.

As one way of overcoming this problem, specimens where a large abnormal clone was detected cytogenetically can be preferentially used over those with less prevalent clones. An alternative approach is to isolate tumor cells from normal cells by dissection, before DNA extraction and CGH analysis. For example, laser capture microdissection, a technique whereby a selected subset of cells are microscopically dissected, can be used to isolate tumor cells. Although somewhat labor intensive, this is the technology that is most likely to eliminate the concern regarding detection of genetic heterogeneity.

Comparative genomic hybridization (CGH) is a well-established technique for surveying the entire genome for abnormalities. However, standard CGH has relatively low resolution and has been used primarily on cell lines and in homogeneous populations (sources). Since a nucleic acid array can be constructed from a large number of DNA fragments for example Bacterial Artificial Chromosome (BAC) clones a Genomic Microarray (GM) can be produced as an article of manufacture that provides a much higher-resolution analysis of chromosomal DNA gains/losses, and has recently shown promise in the analysis of liver cancer following tissue dissection.

One aspect of the invention provides a method to identify genomic aberrations as diagnosis/prognosis markers for certain diseases of interest. Briefly, genomic regions consistently mutated in various disease samples are identified using DNA hybridization with a genomic microarray-comparative genomic hybridization (GM-CGH). Statistical correlation between a subset of the identified genomic aberrations with certain clinically useful data, such as disease onset, progression, and likely clinical outcome are then established. Once identified, the specific subset of genomic aberrations serve as useful markers for reliable and cost-effective diagnosis and/or prognosis means for the disease of interest. These identified disease markers may be provided as specifically designed genomic microarrays in a diagnostic/prognostic test kit, optionally with instructions for using such genomic microarrays (including assay protocols and conditions), and/or control samples and result interpretation.

### Definitions

A chemical “array,” unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm² or even less than 10 cm². For example, features may have widths (that is, diameter, for a round spot) in the range of from about 10 to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in representative embodiments, known. An array feature is generally homogeneous in composition and concentration and the features may be separated by intervening spaces (although arrays without such separation can be fabricated)

In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other (thus, either one could be an unknown mixture of polynucleotides to be detected by binding with the other). “Addressable sets of probes” and analogous terms refer to the multiple regions of different moieties supported by or intended to be supported by the array surface.

The term “sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiment, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

A “test sample” as applied to CGH analysis, refers to a sample that is being analyzed to evaluate DNA copy number, for example, to look for the presence of genetic anomalies, or species differences, for example. A “reference sample” as applied to CGH analysis, is a sample (e.g., a cell or tissue sample) of the same type as the test sample, but whose quantity or degree of representation is known or sequence identity is known. As used herein, a “reference nucleic acid sample” or “reference nucleic acids” refers to nucleic acids comprising sequences whose quantity or degree of representation (e.g., copy number) or sequence identity is known. Similarly, “reference genomic acids” or a “reference genomic sample” refers to genomic nucleic acids comprising sequences whose quantity or degree of representation (e.g., copy number) or sequence identity is known. A “reference nucleic acid sample” may be derived independently from a “test nucleic acid sample,” i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked into the sample.

A test sample and reference sample may both be contacted to a single array for co-hybridization therewith, wherein log ratios of signals from the two samples can be generated by reading the signals for the test sample on a first channel and reading signals for the reference sample on a two-channel analyzer. Alternatively, the test sample may be hybridized to a first array and the reference sample may be hybridized to a second array that is the same as the first array, and signals from each array may be read, and then compared as log ratios

“CGH” or “Comparative Genomic Hybridization” refers generally to techniques for identification of chromosomal alterations (such as in cancer cells, for example). Using CGH, ratios between tumor or test sample and normal or control sample enable the detection of chromosomal amplifications and deletions of regions that may include oncogenes and tumor suppressive genes, for example.

An “array CGH” refers to an array that can be used to compare DNA samples for relative differences in copy number. In general, an array CGH can be used in any assay in which it is desirable to scan a genome with a sample of nucleic acids. For example, an array CGH can be used in location analysis as described in U.S. Pat. No. 6,410,243, the entirety of which is incorporated herein. In certain aspects, array CGH provides probes for screening or scanning a genome of an organism and comprises probes from a plurality of regions of the genome. In one aspect, the array comprises probe sequences for scanning an entire chromosome arm, wherein probes targets are separated by at least about 500 bp, at least about 1 kbp, at least about 5 kbp, at least about 10 kbp, at least about 25 kbp, at least about 50 kbp, at least about 100 kbp, at least about 250 kbp, at least about 500 kbp and at least about 1 Mbp. In another aspect, the array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

An “image analysis system” is a system to enhance and/or accurately quantitate the intensity differences between and/or among the signals from a hybridization and the background staining differences for more accurate and easier interpretation of results. Image analysis and methods to measure intensity are described, for example, in Hiraoka et al., Science, 238: 36-41 (1987) and Aikens et al., Meth. Cell Biol., 29: 291-313 (1989). In such an image analysis system, it is preferred to use a high quality CCD camera whose intensity response is known to be linear over a wide range of intensities.

A “recurrently altered genomic region” or “RAR” refers to a recurrently altered genomic region on chromosome that is defined in table 1 below as regional copy number changes observed in 15 (20%) or more samples out of 76 HCC samples. The array-CGH data described in this invention are available at website (http://systemsbiology.co.kr/micro/CGH/hepato.htm). The term “RAR-G” and “RAR-L” is defined according to Table 1 below. The term “RAR-G” refers to a genomic gain of RAR (RAR-Gain) and the term “RAR-L” refers to a genomic loss of RAR.

TABLE 1
General characteristics of recurrently altered regions

| | | | | | | Number | |
|---|---|---|---|---|---|---|---|
| RAR | Clone | Chromosome | Map position^{a} | Size (Mb) | Cytoband | of cases | Cancer-related genes |
| G1 | RP11-326G21- | 1 | 142391262-183515789 | 41 | 1q21.1-1q32.1 | 49 | PDZK1, MCL1, ARNT, AF1Q, TPM3, |
| | RP5-936P19 | | | | | | ADAR, RPS27, HAX1, PYGO2, CKS1B, |
| | | | | | | | ADAM15, MUC1, HDGF, CCT3, PRCC, |
| | | | | | | | IFI16, AIM2, USF1, SELP, SELE, LAMC2, |
| | | | | | | | TPR, PTGS2, |
| G2 | RP11-572A16- | 1 | 198883756-244440465 | 46 | 1q32.1-1q44 | 45 | KIF14, ELF3, ATF3, TGFB2, WNT3A, |
| | RP11-438H8 | | | | | | AKT3 |
| G3 | RP11-46C20- | 5 | 27487154-35513594 | 8 | 5p14.1-5p13.2 | 19 | AMACR |
| | CTD-2291F22 | | | | | | |
| G4 | CTB-55A14- | 5 | 167592449-174643724 | 7 | 5q34-5q35.2 | 16 | FGF18 |
| | CTB-73D21 | | | | | | |
| G5 | RP1-136B1- | 6 | 2316508-54856430 | 52 | 6p25.2-6p12.1 | 23 | DEK, ID4, E2F3, PRL, MICA, MICB, |
| | RP11-524H19 | | | | | | HMGA1, NOTCH4, MAPK14, PIM1, |
| | | | | | | | TFEB, CCND3, VEGF |
| G6 | RP5-1091E12- | 7 | 54851795-55154846 | 1 | 7p11.2 | 16 | EGFR |
| | RP11-339F13 | | | | | | |
| G7 | RP5-1057M1- | 7 | 79345845-86861115 | 7 | 7q21.11 | 20 | HGF, DMTF1, ABCB1 |
| | RP11-212B1 | | | | | | |
| G8 | RP5-1059M17- | 7 | 100783053-124638919 | 24 | 7q22.1-7q31.33 | 22 | EPO, EPHB4, PIK3CG, CAV1&2, MET, |
| | RP11-420H19 | | | | | | WNT2 |
| G9 | RP11-167E7- | 8 | 48736257-141551817 | 94 | 8q11.21-8q24.3 | 37 | PRKDC, MCM4, SNAI2, LYN, MOS, |
| | RP11-65A5 | | | | | | PLAG1, COPS5, TPD52, E2F5, MMP16, |
| | | | | | | | NBS1, EIF3S3, MYC, KCNK9, PTK2, |
| | | | | | | | EIF2C2, CCNE2 |
| G10 | RP11-472N13- | 10 | 31849328-33818450 | 2 | 10p11.22 | 15 | MAP3K8, NRP1, |
| | RP11-505N10 | | | | | | |
| G11 | RP11-95C14- | 13 | 91284427-112901415 | 21 | 13q31.3-13q34 | 16 | FGF14, ERCC5, |
| | RP11-265C7 | | | | | | |
| G12 | RP11-515O17- | 17 | 50654233-56847074 | 6 | 17q22-17q23.2 | 16 | HLF, MPO, PPM1D, BCAS3, TBX2 |
| | RP11-332H18 | | | | | | |
| G13 | CTD-2043I16- | 19 | 33293135-36979856 | 3 | 19q12 | 19 | CCNE1 |
| | CTC-416D1 | | | | | | |
| G14 | RP5-852M4- | 20 | 327036-61041280 | 61 | 20p13-20q13.33 | 26 | CDC25B, JAG1, SSTR4, BCL2L1, |
| | RP4-563E14 | | | | | | PLAGL2, DNMT3B, E2F1, MMP24, SRC, |
| | | | | | | | TOP1, MYBL2, MMP9, NCOA3, PTPN1, |
| | | | | | | | ZNF217, STK6, BMP7 |
| L1 | RP1-37J18- | 1 | 4487199-7719107 | 3.3 | 1p36.32-1p36.23 | 18 | CHD5, ICMT, CAMTA1 |
| | RP11-338N10 | | | | | | |
| L2 | RP11-285P3- | 1 | 14486429-15354152 | 0.9 | 1p36.21 | 24 | PRDM2, RIZ, CASP9 |
| | RP4-560M15 | | | | | | |
| L3 | RP11-8J9- | 1 | 46823997-69303906 | 23 | 1p33-1p31.2 | 17 | RAD54L, FAF1, S(T)IL, CDKN2C, TTC4, |
| | RP11-412F21 | | | | | | JUN, ARHI |
| L4 | RP11-118B23- | 1 | 84667459-103923047 | 19 | 1p22.3-1p21.1 | 16 | BCL10, CLCA2, LMO4, GTF2B, TGFBR3, |
| | RP5-1108M17 | | | | | | GFI1, EVI5 |
| L5 | RP11-213L8- | 4 | 172094998-190118103 | 18 | 4q33-4q35.2 | 34 | CASP3, FAT |
| | RP11-553E4 | | | | | | |
| L6 | RP1-273N12- | 6 | 99385861-102294657 | 3 | 6q16.2-6q16.3 | 17 | CCNC, GRIK2 |
| | RP11-347H8 | | | | | | |
| L7 | RP1-84N20- | 6 | 125389372-168197568 | 43 | 6q22.31-6q27 | 16 | CRSP3, PLAGL1, SASH1, LATS1, IGF2R, |
| | RP3-470B24 | | | | | | UNC93A, MLLT4 |
| L8 | RP11-336N16- | 8 | 2898583-34455078 | 31 | 8p23.2-8p12 | 34 | CSMD1, DEFB1, NAT1, NAT2, PSD3, |
| | RP11-75P13 | | | | | | TNFRSF10A, TNFRSF10B, TNFRSF10C, |
| | | | | | | | RHOBTB2 |
| L9 | RP11-48M17- | 9 | 2136329-29639069 | 27 | 9p24.2-9p21.1 | 26 | SMARCA2, MTAP, CDKN2B, CDKN2A, |
| | RP11-48L13 | | | | | | RECK, PAX5 |
| L10 | RP11-276H19- | 9 | 86827119-87654534 | 1 | 9q21.33 | 15 | GAS1, DAPK1 |
| | RP11-65B23 | | | | | | |
| L11 | RP11-92C4- | 9 | 98644250-104754734 | 6 | 9q22.33-9q31.1 | 16 | TGFBR1 |
| | RP11-31J20 | | | | | | |
| L12 | RP11-381K7- | 10 | 112963138-116971219 | 4 | 10q25.2-10q25.3 | 15 | CASP7 |
| | RP11-338L11 | | | | | | |
| L13 | RP11-153M24- | 13 | 27414161-59888914 | 32 | 13q12.2-13q21.2 | 19 | BRCA2, CCNA1, RB1, RFP2, DLEU1, |
| | RP11-359P14 | | | | | | DLEU2, DDX26 |
| L14 | RP11-353N19- | 14 | 91389738-99247779 | 8 | 14q32.12-14q32.2 | 15 | BCL11B |
| | RP11-68I8 | | | | | | |
| L15 | RP11-114I12- | 16 | 6846342-26727359 | 20 | 16p13.2-16p12.1 | 18 | SOCS1, ERCC4 |
| | RP11-142A12 | | | | | | |
| L16 | RP11-325K4- | 16 | 55369591-84922042 | 29 | 16q13-16q24.1 | 28 | CDH1, CDH3, BCAR1, WWOX, CDH13, |
| | RP11-514D23 | | | | | | WFDC1 |
| L17 | RP11-135N5- | 17 | 2312021-16718826 | 14 | 17p13.3-17p12 | 39 | TP53 |
| | RP11-219A15 | | | | | | |
| L18 | RP1-270M7- | 21 | 15134621-39788380 | 14 | 21q11.2-21q22.2 | 15 | ADAMTS1 |
| | RP5-1031P17 | | | | | | |

^{a}The mapping position refers to the UCSC genome browser (http://genome.ucsc.edu/; May 2004 freeze)

The purpose of present invention is to provide new diagnostic method and diagnostic kit for determining the prognosis of liver cancer; and cancer related genes using thereof.

To achieve above purpose, the present invention provides a diagnostic methods for determining the prognosis of hepatocellular carcinoma (HCC) comprising the steps of; (a) observing recurrently altered genomic region on a chromosome; (b) measuring variation of one or more of RAR expression variations selected from the RAR variation group consists of gains of RAR-G1 to RAR-G14 and losses of RAR-L1 to RAR-L18 as defined table 1 above. More specifically, the present invention provides diagnostic methods for determining the prognosis of hepatocellular carcinoma (HCC), wherein the RAR expression variation is RAR-G1, RAR-G2, RAR-L17, RAR-G9, RAR-L5, and RAR-L8 as defined according to the table 1. Further specifically, the present invention provides a diagnostic method for determining the tumor stage of hepatocellular carcinoma by using RAR-L2 and RAR-L4, a diagnostic method for prognosis of microvascular invasion of hepatocellular carcinoma by using RAR-G9, RAR-G12, RAR-G13, and RAR-L3, and a diagnostic method for determining the portal vein invasion of the hepatocellular carcinoma by using RAR-G13, RAR-L7 and RAR-L12.

One aspect of the present invention, it provides a diagnostic kit for determining the prognosis of hepatocellular carcinoma (HCC) comprising: (a) a microarray comprising a probe for measuring variation of one or more of RAR expression variations selected from the RAR variation group consists of gains of RAR-G1 to RAR-G14 and losses of RAR-L1 to RAR-L18 as defined table 1 above for observing recurrently altered genomic region (RAR) on a chromosome; and (b) an image analysis device for measuring variation of specific genes expression on the RAR. Specifically, the present invention is a diagnostic kit for determining the prognosis of hepatocellular carcinoma (HCC), wherein the probe for measuring is RAR-G1, RAR-G2, RAR-L17, RAR-G9, RAR-L5 and RAR-L8.

One aspect of the present invention, it provides one or more of genes for diagnosis of hepatocellular carcinoma, selected from the group consists of tropomyosin 3 (TPM3), ribosomal protein S27 (RPS27), hematopoietic cell-specific Lyn substrate 1-associated protein X-1 (HAX1), pygopus homolog 2 (PYGO2), CDC28 protein kinase regulatory subunit 1 (BCKS1B), a disintegrin and metalloprotease 15 (ADAM15), chaperonin subunit 3 (CCT3), papillary renal cell carcinoma (PRCC), kinesin family member 14 (KIF14), Eukaryotic initiation factor 3 (ELF3), transforming Growth Factor beta 2 (TGFB2) and protein kinase B gamma (AKT3).

One aspect of the present invention, it provides a diagnostic method for determining the prognosis of hepatocellular carcinoma (HCC) comprising the step of measuring variation of one or more of gene expression variations selected from the group consists of tropomyosin 3 (TPM3), ribosomal protein S27 (RPS27), hematopoietic cell-specific Lyn substrate 1-associated protein X-1(HAX1), pygopus homolog 2 (PYGO2), CDC28 protein kinase regulatory subunit 1 (BCKS1B), a disintegrin and metalloprotease 15 (ADAM15), chaperonin subunit 3 (CCT3), papillary renal cell carcinoma (PRCC), kinesin family member 14 (KIF14), Eukaryotic initiation factor 3 (ELF3), transforming Growth Factor beta 2 (TGFB2) and protein kinase B gamma (AKT3).

### EXAMPLES

This invention is further illustrated by the following examples which should not be construed as limiting. Reasonable variations and/or modifications of the protocols by a skilled artisan may be used for different experiments, which variations and modifications are within the scope of the instant invention. The contents of all references, patents and published patent applications cited throughout this application, as well as the Figures are hereby incorporated by reference.

### Example 1

### Investigation of Characters of Chromosomal Change in Liver Cancer

(1) Study Materials

Frozen tissues (tumor and adjacent normal tissue pairs) were obtained from 76 primary HCC patients (65 males and 11 females) who underwent surgical resection. This study was performed under the approval of the Institutional Review Board of the Catholic University Medical College of Korea. Tumor stage was determined according to the standard tumor-node-metastasis classification of AJCC guidelines (6th edition). Clinicopathologic information about the 76 cases is available in Table 2. Ten μm-thick frozen sections were prepared and tumor cell-rich areas (tumor cells in more than 60% of the selected area) were microdissected, from which genomic DNA was extracted as described previously.^{11,12}

TABLE 2
Clinicopathologic information of 76 HCC patients

| Case ID | Age (yrs) | Sex | Grade | Stage | Size (mm)^{a} | MVI | PVI | Cap | Virus |
|---|---|---|---|---|---|---|---|---|---|
| HCC1 | 37 | M | 2 | 2 | 57 | 0 | 0 | 1 | B |
| HCC2 | 49 | M | 2 | 2 | 25 | 0 | 0 | 0 | B |
| HCC3 | 42 | F | 3 | 2 | 60 | 0 | 0 | 1 | B |
| HCC4 | 35 | M | 3 | 3 | 80 | 1 | 0 | 0 | B |
| HCC5 | 38 | M | 1 | 1 | 15 | 0 | 0 | — | B |
| HCC6 | 42 | M | 4 | 4 | 55 | 1 | 1 | 0 | B |
| HCC7 | 51 | M | 3 | 3 | 40 | 1 | 0 | 0 | B |
| HCC8 | 58 | M | 3 | 3 | 35 | 1 | 0 | 0 | — |
| HCC9 | 36 | F | 1 | 2 | 30 | 0 | 0 | — | — |
| HCC10 | 54 | F | 3 | 3 | 35 | 1 | 0 | 0 | B |
| HCC11 | 52 | M | 4 | 3 | 70 | 0 | 0 | 1 | B |
| HCC12 | 61 | M | 3 | 3 | 140 | 1 | 0 | 0 | B |
| HCC13 | 52 | M | 2 | 3 | 55 | 1 | 0 | 0 | B |
| HCC14 | 59 | F | 2 | 1 | 15 | 0 | 0 | 1 | B |
| HCC15 | 89 | M | 2 | 2 | 70 | 0 | 0 | 1 | C |
| HCC16 | 56 | M | 3 | 3 | 90 | 1 | 0 | 1 | B |
| HCC17 | 38 | M | 3 | 1 | 15 | 0 | 0 | 0 | B |
| HCC18 | 50 | M | 2 | 2 | 35 | 0 | 0 | 1 | B |
| HCC19 | 56 | F | 2 | 4 | 30 | 1 | 1 | 0 | B |
| HCC20 | 39 | M | 2 | 3 | 25 | 1 | 0 | 0 | B |
| HCC21 | 40 | M | 3 | 4 | 130 | 1 | 1 | 0 | B |
| HCC22 | 53 | M | 3 | 2 | 50 | 0 | 0 | 1 | B |
| HCC23 | 45 | M | 3 | 2 | 15 | 1 | 0 | 1 | B |
| HCC24 | 59 | M | 2 | 2 | 32 | 0 | 0 | 1 | B |
| HCC25 | 55 | M | 2 | 2 | 105 | 0 | 0 | 1 | B |
| HCC26 | 58 | M | 2 | 3 | 44 | 1 | 0 | 1 | B |
| HCC27 | 56 | M | 2 | 2 | 55 | 0 | 0 | 0 | — |
| HCC28 | 43 | M | 2 | 2 | 20 | 1 | 0 | 1 | B |
| HCC29 | 47 | M | 2 | 2 | 35 | 0 | 0 | 1 | C |
| HCC30 | 49 | M | 2 | 2 | 27 | 0 | 0 | 1 | B |
| HCC31 | 53 | M | 2 | 2 | 40 | 0 | 0 | 1 | B |
| HCC32 | 51 | M | 2 | 2 | 55 | 0 | 0 | 1 | B |
| HCC33 | 44 | M | 3 | 2 | 15 | 1 | 0 | 1 | B |
| HCC34 | 66 | M | 2 | 1 | 20 | 0 | 0 | 1 | B |
| HCC35 | 57 | M | 3 | 3 | 26 | 1 | 0 | 1 | B |
| HCC36 | 38 | M | 2 | 2 | 45 | 0 | 0 | 1 | B |
| HCC37 | 55 | M | 2 | 2 | 70 | 0 | 0 | 1 | B |
| HCC38 | 42 | M | 2 | 3 | 35 | 1 | 0 | 1 | B |
| HCC39 | 51 | F | 2 | 2 | 30 | 0 | 0 | 1 | B |
| HCC40 | 55 | M | 1 | 2 | 25 | 0 | 0 | 1 | B |
| HCC41 | 43 | M | 4 | 3 | 50 | 1 | 0 | 1 | B |
| HCC42 | 57 | M | 3 | 3 | 55 | 1 | 0 | 0 | B |
| HCC43 | 37 | M | 3 | 2 | 20 | 1 | 0 | 0 | B |
| HCC44 | 65 | M | 3 | 3 | 28 | 1 | 0 | 1 | B |
| HCC45 | 55 | M | 2 | 3 | 60 | 1 | 0 | 0 | B |
| HCC46 | 60 | M | 2 | 2 | 70 | 0 | 0 | 1 | B |
| HCC47 | 43 | M | 4 | 3 | 230 | 1 | 0 | 0 | B |
| HCC48 | 31 | M | 2 | 1 | 20 | 0 | 0 | 0 | B |
| HCC49 | 66 | M | 1 | 2 | 28 | 0 | 0 | 0 | B |
| HCC50 | 62 | F | 1 | 2 | 31 | 0 | 0 | 1 | B |
| HCC51 | 46 | M | 2 | 3 | 17 | 1 | 0 | 1 | B |
| HCC52 | 52 | F | 2 | 1 | 20 | 0 | 0 | 0 | B |
| HCC53 | 49 | M | 2 | 3 | 110 | 0 | 0 | 1 | B |
| HCC54 | 45 | M | 1 | 3 | 30 | 1 | 0 | 0 | B |
| HCC55 | 52 | M | 3 | 2 | 35 | 1 | 1 | 1 | B |
| HCC56 | 75 | M | 3 | 3 | 120 | 0 | 0 | 1 | — |
| HCC57 | 42 | M | 1 | 2 | 30 | 0 | 0 | 1 | B |
| HCC58 | 49 | M | 3 | 3 | 90 | 1 | 0 | 1 | B |
| HCC59 | 61 | M | 4 | 4 | 60 | 1 | 1 | 1 | B |
| HCC60 | 52 | M | 4 | 2 | 35 | 1 | 0 | 1 | B |
| HCC61 | 41 | M | 2 | 3 | 50 | 1 | 1 | 1 | B |
| HCC62 | 71 | M | 2 | 3 | 45 | 1 | 1 | 1 | B |
| HCC63 | 43 | M | 3 | 2 | 35 | 1 | 0 | 1 | B |
| HCC64 | 51 | M | 4 | 3 | 30 | 1 | 1 | 1 | B |
| HCC65 | 58 | F | 2 | 3 | 67 | 0 | 1 | 1 | — |
| HCC66 | 42 | M | 4 | 4 | 80 | 1 | 1 | 1 | B |
| HCC67 | 48 | M | 2 | 2 | 25 | 0 | 1 | 1 | B |
| HCC68 | 48 | M | 1 | 2 | 20 | 0 | 0 | 0 | B |
| HCC69 | 29 | F | 2 | 3 | 60 | 0 | 0 | 1 | B |
| HCC70 | 52 | M | 2 | 3 | 15 | 0 | 0 | 0 | B |
| HCC71 | 51 | M | 4 | 3 | 55 | 1 | 1 | 1 | B |
| HCC72 | 64 | F | 4 | 3 | 60 | 0 | 0 | 1 | B |
| HCC73 | 68 | M | 1 | 3 | 35 | 0 | 0 | 1 | — |
| HCC74 | 49 | M | 2 | 3 | — | 0 | 0 | 0 | B |
| HCC75 | 68 | M | 3 | 3 | 110 | 1 | 1 | 1 | — |
| HCC76 | 48 | M | 2 | 2 | 20 | 0 | 1 | 1 | B |

^{a}If multiple masses are observed, the size of the largest one is used to as tumor size of the corresponding case.
MVI, microvascular invasion;
PVI, portal vein invasion;
Cap, encapsulation

(2) Array Comparative Genomic Hybridization

A large insert clone array covering the entire human genome at 1 Mb resolution was used for profiling genomic alterations.¹³ Array-CGH was performed as described elsewhere using MAUI hybridization station (BioMicro Systems, Salt Lake city, Utah).^{11,12} Data processing, normalization, and re-aligning of raw array-CGH data were performed using web-based array-CGH analysis software ArrayCyGHt (http://genomics.catholic.ac.kr/arrayCGH/).¹⁴ We used print-tip loess normalization method for analysis. Large insert clones (n=2,958) and genomic coordinates such as cytogenetic bands or gene positions were mapped according to the UCSC genome browser (http://genome.ucsc.edu/; May 2004 freeze).

(3) Statistical Analysis

To see the association between chromosomal changes such as RARs and total number of altered clones, and clinicopathologic phenotypes, eight clinical parameters were treated as categorical variables; age (<35 years versus ≧35 years), sex, grade (grade 1 and 2 as low versus 3 and 4 as high), stage (stage I and II as early versus stage III and IV as advanced), tumor size (<3.5 cm versus ≧3.5 cm) along with the presence or absence of microvascular invasion, portal vein invasion, and encapsulation. Significance of the different distribution of RARs in each category was tested by Chi squared or two-sided Fisher's exact test. The mean number of altered clones in 8 categories and expression levels of genes in different RAR groups were compared by independent t-test. Stata version 9,1 (Stata Corporation, Texas) was used and P value less than 0.05 was considered significant in all statistical analyses.

(4) Results

Profiles of Chromosomal Alterations

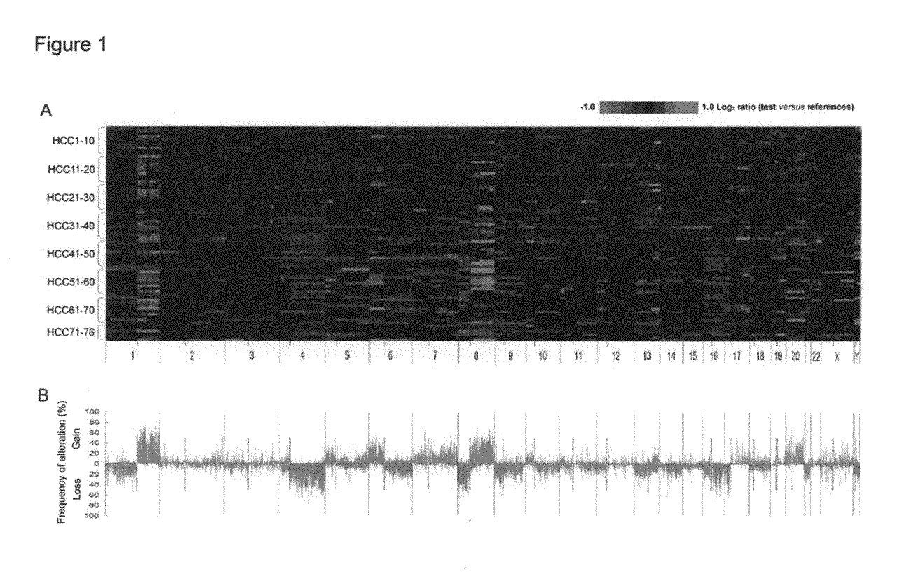

The overall profile of chromosomal alterations identified in the 76 primary HCCs is illustrated in FIG. 1A. Chromosomal alterations are not randomly distributed along the genome but clustered in certain chromosomal segments (FIG. 1B). Mean number of altered clones per case is 677.1 (338.7 clones gained and 338.4 clones lost) out of total 2,958 clones. In other words, 22.9% (11.5% gained and 11.4% lost) of the whole genome is altered per each case on average. Mean numbers of altered clones are significantly higher in high-grade tumors than low-grade ones (769.9 versus 594.6 clones; P=0.002), in microinvasion positive cases than negative ones (764.1 versus 598.9 clones; P=0.016) and in bigger tumors than smaller ones (756.1 versus 602.3 clones; P=0.026). In case of entire chromosomal arm changes, five chromosomal gains in 1q, 6p, 8q, 20p, and 20q and 5 losses in 4q, 8p, 9p, 16q, and 17p are repetitively observed in over 30% of the samples. The alteration frequency of chromosomal arms is summarized in Table 3.

TABLE 3
Frequencies of entire chromosomal arm changes in HCC

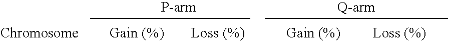

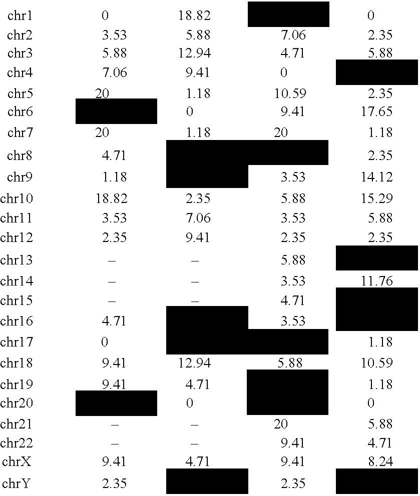

The recurrent changes observed more than 20% of samples are shown as shaded.

### Recurrently Altered Regions in HCC

Although entire chromosomal arm changes appeared occasionally, vast majority of copy number alterations in HCCs are localized regional changes. To delineate the frequently observed consensus-regions, we defined the RARs which are regional chromosomal alterations observed in at least 15 cases (20% of HCC cases) among the 76 HCCs. In total, 14 RAR gains (RAR-G) and 18 RAR losses (RAR-L) were detected (Table 1). Five most frequent RARs are RAR-G1 (64.5%: 1q21.1-1q32.1), RAR-G2 (59.2%: 1q32.1-1q44), RAR-L17 (51.3%: 17p13.3-17p12), RAR-G9 (48.7%: 8q11.21-8q24.3) and RAR-L5/L8 (both 44.7%: 4q33-4q35.2/8p23.2-8p12). Average size of RAR-Gs (26.6 Mb; ranged 1-94 Mb) is larger than that of RAR-Ls (16.5 Mb; ranged 0.9-43 Mb). Well-known oncogenes such as MYC, FGF, EGFR, and CCND3 along with tumor suppressor genes such as TP53, RB1, CDKN2A, and CDKN2B are located in identified RARs. The other genes in the RARs are also thought to be potential cancer-related genes contributing to hepatocarcinogenesis directly or indirectly (Table 1).

TABLE 1
General characteristics of recurrently altered regions

| | | | | | | Number | |
|---|---|---|---|---|---|---|---|
| RAR | Clone | Chromosome | Map position^{a} | Size (Mb) | Cytoband | of cases | Cancer-related genes |
| G1 | RP11-326G21- | 1 | 142391262-183515789 | 41 | 1q21.1-1q32.1 | 49 | PDZK1, MCL1, ARNT, AF1Q, TPM3, |
| | RP5-936P19 | | | | | | ADAR, RPS27, HAX1, PYGO2, CKS1B, |
| | | | | | | | ADAM15, MUC1, HDGF, CCT3, PRCC, |
| | | | | | | | IFI16, AIM2, USF1, SELP, SELE, LAMC2, |
| | | | | | | | TPR, PTGS2, |
| G2 | RP11-572A16- | 1 | 198883756-244440465 | 46 | 1q32.1-1q44 | 45 | KIF14, ELF3, ATF3, TGFB2, WNT3A, |
| | RP11-438H8 | | | | | | AKT3 |
| G3 | RP11-46C20- | 5 | 27487154-35513594 | 8 | 5p14.1-5p13.2 | 19 | AMACR |
| | CTD-2291F22 | | | | | | |
| G4 | CTB-55A14- | 5 | 167592449-174643724 | 7 | 5q34-5q35.2 | 16 | FGF18 |
| | CTB-73D21 | | | | | | |
| G5 | RP1-136B1- | 6 | 2316508-54856430 | 52 | 6p25.2-6p12.1 | 23 | DEK, ID4, E2F3, PRL, MICA, MICB, |
| | RP11-524H19 | | | | | | HMGA1, NOTCH4, MAPK14, PIM1, |
| | | | | | | | TFEB, CCND3, VEGF |
| G6 | RP5-1091E12- | 7 | 54851795-55154846 | 1 | 7p11.2 | 16 | EGFR |
| | RP11-339F13 | | | | | | |
| G7 | RP5-1057M1- | 7 | 79345845-86861115 | 7 | 7q21.11 | 20 | HGF, DMTF1, ABCB1 |
| | RP11-212B1 | | | | | | |
| G8 | RP5-1059M17- | 7 | 100783053-124638919 | 24 | 7q22.1-7q31.33 | 22 | EPO, EPHB4, PIK3CG, CAV1&2, MET, |
| | RP11-420H19 | | | | | | WNT2 |
| G9 | RP11-167E7- | 8 | 48736257-141551817 | 94 | 8q11.21-8q24.3 | 37 | PRKDC, MCM4, SNAI2, LYN, MOS, |
| | RP11-65A5 | | | | | | PLAG1, COPS5, TPD52, E2F5, MMP16, |
| | | | | | | | NBS1, EIF3S3, MYC, KCNK9, PTK2, |
| | | | | | | | EIF2C2, CCNE2 |
| G10 | RP11-472N13- | 10 | 31849328-33818450 | 2 | 10p11.22 | 15 | MAP3K8, NRP1, |
| | RP11-505N10 | | | | | | |
| G11 | RP11-95C14- | 13 | 91284427-112901415 | 21 | 13q31.3-13q34 | 16 | FGF14, ERCC5, |
| | RP11-265C7 | | | | | | |
| G12 | RP11-515O17- | 17 | 50654233-56847074 | 6 | 17q22-17q23.2 | 16 | HLF, MPO, PPM1D, BCAS3, TBX2 |
| | RP11-332H18 | | | | | | |
| G13 | CTD-2043I16- | 19 | 33293135-36979856 | 3 | 19q12 | 19 | CCNE1 |
| | CTC-416D1 | | | | | | |
| G14 | RP5-852M4- | 20 | 327036-61041280 | 61 | 20p13-20q13.33 | 26 | CDC25B, JAG1, SSTR4, BCL2L1, |
| | RP4-563E14 | | | | | | PLAGL2, DNMT3B, E2F1, MMP24, SRC, |
| | | | | | | | TOP1, MYBL2, MMP9, NCOA3, PTPN1, |
| | | | | | | | ZNF217, STK6, BMP7 |
| L1 | RP1-37J18- | 1 | 4487199-7719107 | 3.3 | 1p36.32-1p36.23 | 18 | CHD5, ICMT, CAMTA1 |
| | RP11-338N10 | | | | | | |
| L2 | RP11-285P3- | 1 | 14486429-15354152 | 0.9 | 1p36.21 | 24 | PRDM2, RIZ, CASP9 |
| | RP4-560M15 | | | | | | |
| L3 | RP11-8J9- | 1 | 46823997-69303906 | 23 | 1p33-1p31.2 | 17 | RAD54L, FAF1, S(T)IL, CDKN2C, TTC4, |
| | RP11-412F21 | | | | | | JUN, ARHI |
| L4 | RP11-118B23- | 1 | 84667459-103923047 | 19 | 1p22.3-1p21.1 | 16 | BCL10, CLCA2, LMO4, GTF2B, TGFBR3, |
| | RP5-1108M17 | | | | | | GFI1, EVI5 |
| L5 | RP11-213L8- | 4 | 172094998-190118103 | 18 | 4q33-4q35.2 | 34 | CASP3, FAT |
| | RP11-553E4 | | | | | | |
| L6 | RP1-273N12- | 6 | 99385861-102294657 | 3 | 6q16.2-6q16.3 | 17 | CCNC, GRIK2 |
| | RP11-347H8 | | | | | | |
| L7 | RP1-84N20- | 6 | 125389372-168197568 | 43 | 6q22.31-6q27 | 16 | CRSP3, PLAGL1, SASH1, LATS1, IGF2R, |
| | RP3-470B24 | | | | | | UNC93A, MLLT4 |
| L8 | RP11-336N16- | 8 | 2898583-34455078 | 31 | 8p23.2-8p12 | 34 | CSMD1, DEFB1, NAT1, NAT2, PSD3, |
| | RP11-75P13 | | | | | | TNFRSF10A, TNFRSF10B, TNFRSF10C, |
| | | | | | | | RHOBTB2 |
| L9 | RP11-48M17- | 9 | 2136329-29639069 | 27 | 9p24.2-9p21.1 | 26 | SMARCA2, MTAP, CDKN2B, CDKN2A, |
| | RP11-48L13 | | | | | | RECK, PAX5 |
| L10 | RP11-276H19- | 9 | 86827119-87654534 | 1 | 9q21.33 | 15 | GAS1, DAPK1 |
| | RP11-65B23 | | | | | | |
| L11 | RP11-92C4- | 9 | 98644250-104754734 | 6 | 9q22.33-9q31.1 | 16 | TGFBR1 |
| | RP11-31J20 | | | | | | |
| L12 | RP11-381K7- | 10 | 112963138-116971219 | 4 | 10q25.2-10q25.3 | 15 | CASP7 |
| | RP11-338L11 | | | | | | |
| L13 | RP11-153M24- | 13 | 27414161-59888914 | 32 | 13q12.2-13q21.2 | 19 | BRCA2, CCNA1, RB1, RFP2, DLEU1, |
| | RP11-359P14 | | | | | | DLEU2, DDX26 |
| L14 | RP11-353N19- | 14 | 91389738-99247779 | 8 | 14q32.12-14q32.2 | 15 | BCL11B |
| | RP11-68I8 | | | | | | |
| L15 | RP11-114I12- | 16 | 6846342-26727359 | 20 | 16q13.2-16p12.1 | 18 | SOCS1, ERCC4 |
| | RP11-142A12 | | | | | | |
| L16 | RP11-325K4- | 16 | 55369591-84922042 | 29 | 16q13-16q24.1 | 28 | CDH1, CDH3, BCAR1, WWOX, CDH13, |
| | RP11-514D23 | | | | | | WFDC1 |
| L17 | RP11-135N5- | 17 | 2312021-16718826 | 14 | 17p13.3-17p12 | 39 | TP53 |
| | RP11-219A15 | | | | | | |
| L18 | RP1-270M7- | 21 | 15134621-39788380 | 14 | 21q11.2-21q22.2 | 15 | ADAMTS1 |
| | RP5-1031P17 | | | | | | |

^{a}The mapping position refers to the UCSC genome browser (http://genome.ucsc.edu/; May 2004 freeze)

### RARs and Clinical Characteristics

Eight types of clinical variables (age of onset, sex, grade, stage, size, portal vein invasion, microvascular invasion and encapsulation) were analyzed for their associations with the RARs (Table 4). RARs and associated characteristics are as follows; RAR-L11 and -L16 with early onset of age, RAR-G5, -G7, -G8 and RAR-L13 with male sex, RAR-L5, -L9, -L10, -L11 and -L13 with high tumor grade, especially RAR-L10 showing highly significant association, RAR-L2 and -L4 with advanced tumor stage, RAR-G9, -G12, -G13 and RAR-L3 with microvascular invasion, RAR-G13, RAR-L7 and -L12 with portal vein invasion in negative direction, RAR-G4 with larger tumor size, and RAR-G6 and -G7 with encapsulation.

TABLE 4
Association between RARs and clinical features

| | | Early onset (<50) | Late onset (≧50) | Total | P value |
|---|---|---|---|---|---|
| RAR-L11 | − | 23 | 37 | 60 | 0.030 |
| | + | 11 | 5 | 16 | |
| RAR-L16 | − | 23 | 37 | 60 | 0.030 |
| | + | 11 | 5 | 16 | |
| | | Female | Male | Total | P value |
| RAR-G5 | − | 11 | 42 | 53 | 0.018 |
| | + | 0 | 23 | 23 | |
| RAR-G7 | − | 11 | 45 | 56 | 0.032 |
| | + | 0 | 20 | 20 | |
| RAR-G8 | − | 11 | 43 | 54 | 0.022 |
| | + | 0 | 22 | 22 | |
| RAR-L13 | − | 11 | 46 | 57 | 0.038 |
| | + | 0 | 19 | 19 | |
| | | Low grade (1, 2) | High grade (3, 4) | Total | P value |
| RAR-L5 | − | 30 | 12 | 42 | 0.016 |
| | + | 15 | 19 | 34 | |
| RAR-L9 | − | 36 | 14 | 42 | 0.002 |
| | + | 9 | 17 | 26 | |
| RAR-L10 | − | 42 | 19 | 51 | 0.001 |
| | + | 3 | 12 | 15 | |
| RAR-L11 | − | 40 | 20 | 60 | 0.01 |
| | + | 5 | 11 | 16 | |
| RAR-L13 | − | 38 | 19 | 57 | 0.022 |
| | + | 7 | 12 | 19 | |
| | | Stage (I, II) | Stage (III, IV) | Total | P value |
| RAR-L2 | − | 30 | 22 | 52 | 0.048 |
| | + | 8 | 16 | 24 | |
| RAR-L4 | − | 34 | 26 | 60 | 0.024 |
| | + | 4 | 12 | 16 | |
| | | Size (<3.5 cm) | Size (≧3.5 cm) | Total | P value |
| RAR-G4 | − | 35 | 25 | 60 | 0.018 |
| | + | 4 | 12 | 16 | |
| | | MVI (−) | MVI (+) | Total | P value |
| RAR-G9 | − | 27 | 12 | 39 | 0.003 |
| | + | 13 | 24 | 37 | |
| RAR-G12 | − | 36 | 24 | 60 | 0.013 |
| | + | 4 | 12 | 16 | |
| RAR-G13 | − | 25 | 32 | 57 | 0.008 |
| | + | 15 | 4 | 19 | |
| | | PVI (−) | PVI (+) | Total | P value |
| RAR-G13 | − | 43 | 14 | 57 | 0.017 |
| | + | 19 | 0 | 19 | |
| RAR-L7 | − | 46 | 14 | 60 | 0.032 |
| | + | 16 | 0 | 16 | |
| RAR-L12 | − | 48 | 14 | 62 | 0.049 |
| | + | 14 | 0 | 14 | |
| | | Cap (−) | Cap (+) | Total | P value |
| RAR-G6 | − | 22 | 36 | 60 | 0.054 |
| | + | 2 | 14 | 16 | |
| RAR-G7 | − | 21 | 33 | 54 | 0.051 |
| | + | 3 | 17 | 20 | |

Note:
MVI, microvascular invasion;
PVI, portal vein invasion;
Cap, encapsulation

### Example 2

### Analysis of Recurrently Altered Genomic Region Having Clinically Importance

### (1) Functional Enrichment Analysis

Functional enrichment analysis based on gene ontology was performed for the RARs significantly associated with clinicopatholigical characteristics. In brief, gene sets for enrichment analysis were prepared using 17,661 known genes with genomic coordinates downloaded from the UCSC genome browser (2004, May Freeze). Genes were grouped into specific sets using NetAffx Gene Ontology Mining Tools according to functions annotated in public gene databases such as GO (Gene Ontology), KEGG (Kyoto Encyclopedia of Genes and Genomes) and GenMAPP (Gene Map Annotator and Pathway Profiler).¹⁶⁻¹⁹ A total of 1,632 gene sets were prepared for enrichment analysis. The functional enrichment analysis was performed using GEAR software (http://systemsbiology.co.kr/GEAR/).²⁰ The significance of enrichment was calculated by hypergeometric distribution and P value less than 0.01 was considered significant.

### (2) Identification of Copy Number Alteration

To set the cutoff value for chromosomal alterations of individual clones based on standard deviation (SD), we performed six independent hybridizations using DNA from normal individuals (four sex-matched and two male-to-female), from which control SD values of individual clones were obtained. Chromosomal gain or loss was assigned when the normalized log₂ intensity ratio of each data point exceeded or fell below ±3 SD derived from normal control hybridizations. Regional copy number change was defined as DNA copy number alteration stretching over 2 or more consecutive large insert clones, but not across an entire chromosomal arm. High-level amplification of clones was defined when their intensity ratios were higher than 1.0 in log₂ scale, and vice versa for homozygous deletion. RAR was defined as regional copy number changes observed in 15 (20%) or more samples out of 76 HCC samples. The array-CGH data described in this study are available at website (http://systemsbiology.co.kr/micro/CGH/hepato.htm).

### (3) Real-Time Quantitative PCR Assay

The first-strand cDNA was synthesized from total RNA of 20 HCC and normal tissue pairs using M-MLV reverse transcriptase (Invitrogen, Carlsbad, Calif.). Real-time quantitative PCR was performed using Mx3000P qPCR system and MxPro Version 3.00 software (Stratagene, Calif., USA). Twenty μl of real-time qPCR mixture contains 10 ng of cDNA, 1×SYBR® Green Tbr polymerase mixture (FINNZYMES, Finland), 1×ROX, and 20 pmole of primers. GAPDH was used as an internal control in each procedure. Thermal cycling was done as follows: 10 min at 95° C. followed by 40 cycles of 10 sec at 94° C., 30 sec at 53-58° C. and 30 sec at 72° C. To verify specific amplification, melting curve analysis was performed (55-95° C., 0.5° C./sec). Relative quantification was performed by the ΔΔCT method.¹⁵ All the experiments were repeated twice and mean value of intensity ratios with SD was plotted for each case. Primer sequences for TPM3, RPS27, HAX1, PYGO2, CKS1B, ADAM15, CCT3, PRCC, KIF14, ELF3, TGFB2, and AKT3 are available in Table 5.

TABLE 5
Primers used for candidate gene-specific RT-PCR located in chromosone 1q

| Gene | Size | | |
|---|---|---|---|
| Symbol | (bp) | Sequence (Forward) | Sequence (Reverse) |
| TPM3 | 141 | 5-GAG AGG TAT GAA GGT TAT TGA-3 | 5-ATCACCAACTTACGAGCCACC-3′ |
| | | (Seq. ID NO. 1) | (Seq. ID NO. 2) |
| RPS27 | 161 | 5-CTT TCC GGC GGT GAC GAC-3 | 5-TTT TAT AGC ATC CTG GGC ATT TC-3 |
| | | (Seq. ID NO. 3) | (Seq. ID NO. 4) |
| HAX1 | 200 | 5-GTA GGG CCG GAC AGA GAC TAC AG-3 | 5-GTG GGC AAT GGG TGA GAG GTG-3 |
| | | (Seq. ID NO. 5) | (Seq. ID NO. 6) |
| PYGO2 | 193 | 5-AGG GCC CTG CAT ACT CAC ATC TG-3 | 5-CCC CCT GCA CAC GGA AGC-3 |
| | | (Seq. ID NO. 7) | (Seq. ID NO. 8) |
| CKS1B | 79 | 5-CTT GGC GTT CAG CAG AGT CAG G-3 | 5-GGC GCC GGA ACA GCA AGA T-3 |
| | | (Seq. ID NO. 9) | (Seq. ID NO. 10) |
| ADAM15 | 194 | 5-TCC AGC CCC AGC CAA GAC CT-3 | 5-GCT CGC CCG GCT CCA CAA ACA TA-3 |
| | | (Seq. ID NO. 11) | (Seq. ID NO. 12) |
| CCT3 | 119 | 5-GGG TGC GGT GAT TGG CGA CTA C-3 | 5-TGG GGG AAC CGG CAG AAC CT-3 |
| | | (Seq. ID NO. 13) | (Seq. ID NO. 14) |
| PRCC | 166 | 5-TGC CTC CGC CCC CTC AGA TGC T-3 | 5-CTC CCC AAC TCC CGC CGC TTC A-3 |
| | | (Seq. ID NO. 15) | (Seq. ID NO. 16) |
| KIF14 | 221 | 5-CTG CTC TAC GGC TCA CAC TAA TGG-3 | 5-CTG GCA GCG GGA CTA ATC GTA-3 |
| | | (Seq. ID NO. 17) | (Seq. ID NO. 18) |
| ELF3 | 214 | 5-CTC GCC TCC CCA CCC TCC TCT T-3 | 5-GCC CCT GCT CTG TCC TCT CCA TCA-3 |
| | | (Seq. ID NO. 19) | (Seq. ID NO. 20) |
| TGFB2 | 250 | 5-AAT GCC ATC CCG CCC ACT TTC TAC-3 | 5-GCC ATT CGC CTT CTG CTC TTG TTT-3 |
| | | (Seq. ID NO. 21) | (Seq. ID NO. 22) |
| AKT3 | 190 | 5-GAG CCC ACC ATT GTT CAT TTG-3 | 5-GCA CGC CAC CAC CCT TCC-3 |
| | | (Seq. ID NO. 23) | (Seq. ID NO. 24) |
| GAPDH | 301 | 5-GCG GGG CTC TCC AGA ACA TCA-3 | 5-CCA GCC CCA GCG TCA AAG GTG-3 |
| | | (Seq. ID NO. 25) | (Seq. ID NO. 26) |

### (4) Results

### Functional Annotation of the Clinically Significant RARs

We investigated functional categories of the genes enriched in the RARs which showed significant association with clinical features. Table 6 lists the enriched functional pathways in tumor grade-associated RARs. Top 5 pathways have interferon-related gene families in common as member genes, since interferon-loci are included in tumor grade-related RARs. Since all of these RARs are copy number losses, it can be assumed that the 5 interferon-related pathways are repressed. Cell cycle regulation and angiogenesis pathways are also found to be significantly associated with tumor grade-related RARs. Functional enrichment analysis results of other clinical feature-related RARs are available in the Table 7.

TABLE 6
Functional pathways enriched in tumor grade-associated RARs

| | Gene | Observed | | |
|---|---|---|---|---|
| Functional annotations | size^{a} | genes^{b} | P-value^{e} | Genes^{c} |
| hematopoietin/interferon-class | 20 | 14 | 5.25E−21 | IFNA1, IFNA2, IFNA4, IFNA5, IFNA6, |
| cytokine receptor binding | | | | IFNA8, IFNA10, IFNA13, IFNA14, IFNA16, |
| | | | | IFNA17, IFNA21, IFNB1, IFNW1 |
| interferon-alpha/beta receptor | 9 | 9 | 1.18E−16 | IFNA1, IFNA2, IFNA4, IFNA10, IFNA13, |
| binding | | | | IFNA16, IFNA17, IFNB1, IFNW1 |
| response to virus | 66 | 14 | 5.15E−12 | IFN family^{d} |
| defense response | 126 | 15 | 4.52E−09 | IFN family, IFNE1 |
| cytokine activity | 177 | 17 | 1.15E−08 | IFN family, TNFSF11, CER1, IFNE1 |
| physiological process | 18 | 4 | 0.0002 | INSL4, RLN1, RLN2, INSL6 |
| regulation of cyclin dependent | 34 | 4 | 0.0027 | CDKN2A, CDKN2B, CCNA1, RGC32 |
| protein kinase activity | | | | |
| condensed chromosome | 6 | 2 | 0.0042 | HMGB1, HMGB2 |
| Angiogenesis | 41 | 4 | 0.0053 | COL15A1, FLT1, VEGFC, HAND2 |
| Pregnancy | 47 | 4 | 0.0086 | FLT1, INSL4, RLN1, RLN2 |

^{a}number of genes in the functionally annotated gene sets
^{b}number of observed genes in tumor grade-related RARs
^{c}gene symbols for the observed genes.
^{d}includes 14 genes in hematopoietin/interferon-class cytokine receptor binding category. It is used to avoid repeating the gene symbols.
^{e}significance level of enrichment was calculated using hypergeometric distribution and P < 0.01 was considered significant.

TABLE 7
Functional categories enriched in clinicopathologic phenotype-related RARs

| Clinical | | | Observed | | |
|---|---|---|---|---|---|
| phenotypes | Functional annotations | Gene size | genes | P-value | Genes |
| Age | N-acetylglucosamine 6-O- | 5 | 3 | 2.95E−05 | CHST6, CHST4, CHST5 |
| | sulfotransferase activity | | | | |
| | intrinsic to Golgi membrane | 8 | 3 | 0.0002 | CHST6, CHST4, CHST5 |
| | homophilic cell adhesion | 92 | 7 | 0.0004 | CDH1, CDH3, CDH5, CDH8, CDH11, CDH13, |
| | | | | | CDH16 |
| | N-acetylglucosamine metabolism | 11 | 3 | 0.0005 | CHST6, CHST4, CHST5 |
| | sulfur metabolism | 12 | 3 | 0.0006 | CHST6, CHST4, CHST5 |
| | chemotaxis | 104 | 7 | 0.0008 | CCL17, CCL22, CKLF, CMTM1, CMTM3, CMTM4, |
| | | | | | CMTM2 |
| | amino acid-polyamine transporter | 35 | 4 | 0.0016 | SLC12A3, SLC12A4, SLC7A6, FLJ10815 |
| | activity | | | | |
| | aminomethyltransferase activity | 5 | 2 | 0.0020 | GCSH, PDPR |
| | cytoplasmic dynein complex | 5 | 2 | 0.0020 | DYNC1LI2, DYNLRB2 |
| | glycine catabolism | 6 | 2 | 0.00304 | GCSH, PDPR |
| | cation:chloride symporter activity | 6 | 2 | 0.0030 | SLC12A3, SLC12A4 |
| | amino acid transport | 43 | 4 | 0.0034 | SLC12A3, SLC12A4, SLC7A6, FLJ10815 |
| | neuropeptide signaling pathway | 71 | 5 | 0.0037 | AGRP, GPR56, PKD1L2, GPR114, GPR97 |
| | cytokine activity | 177 | 8 | 0.0043 | CCL17, CCL22, CX3CL1, CKLF, CMTM1, CMTM3, |
| | | | | | CMTM4, CMTM2 |
| | chemokine activity | 46 | 4 | 0.0043 | CCL17, CCL22, CX3CL1, CKLF |
| | telomerase-dependent telomere | 9 | 2 | 0.0070 | TERF2, TERF2IP |
| | maintenance | | | | |
| Sex | nucleosome | 73 | 44 | 1.59E−41 | Histone family |
| | nucleosome assembly | 83 | 44 | 4.06E−38 | Histone familiy |
| | chromosome organization and | 87 | 43 | 1.46E−35 | Histone familiy |
| | biogenesis | | | | |
| | chromosome | 102 | 44 | 3.95E−33 | Histone familiy |
| | MHC class II receptor activity | 14 | 13 | 2.23E−17 | HLA-C, HLA-DMA, HLA-DMB, HLA-DOA, HLA- |
| | | | | | DOB, HLA-DPA1, HLA-DPB1, HLA-DQA1, HLA- |
| | | | | | DQA2, HLA-DQB1, HLA-DRA, HLA-DRB1, HLA- |
| | | | | | DRB5 |
| | antigen presentation, exogenous | 13 | 12 | 4.86E−16 | HLA-DMA, HLA-DMB, HLA-DOA, HLA-DOB, HLA- |
| | antigen | | | | DPA1, HLA-DPB1, HLA-DQA1, HLA-DQA2, HLA- |
| | | | | | DQB1, HLA-DRA, HLA-DRB1, HLA-DRB5 |
| | antigen processing, exogenous | 14 | 12 | 3.27E−15 | HLA-DMA, HLA-DMB, HLA-DOA, HLA-DOB, HLA- |
| | antigen via MHC class II | | | | DPA1, HLA-DPB1, HLA-DQA1, HLA-DQA2, HLA- |
| | | | | | DQB1, HLA-DRA, HLA-DRB1, HLA-DRB5 |
| | antigen processing, endogenous | 10 | 9 | 4.82E−12 | HFE, HLA-A, HLA-B, HLA-C, HLA-E, HLA-F, HLA- |
| | antigen via MHC class I | | | | G, TAP2, TAPBP |
| | DNA packaging | 17 | 9 | 8.91E−09 | Histone familiy |
| | antigen presentation, endogenous | 9 | 7 | 9.13E−09 | HFE, HLA-A, HLA-B, HLA-C, HLA-E, HLA-F, HLA-G |
| | antigen | | | | |
| | detection of pest, pathogen or | 7 | 6 | 4.30E−08 | HLA-DMA, HLA-DMB, HLA-DPB1, HLA-DRB1, |
| | parasite | | | | HLA-DRB5, HLA-G |
| | establishment and/or maintenance of | 33 | 11 | 7.37E−08 | Histone familiy |
| | chromatin architecture | | | | |
| | MHC class I protein complex | 16 | 8 | 1.11E−07 | HFE, HLA-A, HLA-B, HLA-C, HLA-E, HLA-F, HLA- |
| | | | | | G, MICB |
| | phosphoinositide-mediated signaling | 23 | 9 | 2.37E−07 | Histone familiy |
| | antigen presentation | 19 | 8 | 5.79E−07 | HFE, HLA-A, HLA-B, HLA-C, HLA-E, HLA-F, HLA- |
| | | | | | G, MICB |
| | MHC class I receptor activity | 21 | 8 | 1.44E−06 | HFE, HLA-A, HLA-B, HLA-C, HLA-E, HLA-F, HLA- |
| | | | | | G, MICB |
| | olfactory receptor activity | 82 | 13 | 4.67E−05 | OR2H2, OR2B6, OR12D2, OR11A1, OR2W1, OR2J2, |
| | | | | | OR2H1, OR5V1, OR2B2, OR12D3, OR2B3, OR5U1, |
| | | | | | OR10C1 |
| | sensory perception of smell | 88 | 13 | 9.91E−05 | OR2H2, OR2B6, OR12D2, OR11A1, OR2W1, OR2J2, |
| | | | | | OR2H1, OR5V1, OR2B2, OR12D3, OR2B3, OR5U1, |
| | | | | | OR10C1 |
| | Proteasome_Degradation | 57 | 9 | 0.0007 | HLA-A, HLA-B, HLA-C, HLA-E, HLA-F, HLA-G, |
| | (GenMAPP) | | | | PSMB8, PSMB9, PSMC2 |
| | fusion of sperm to egg plasma | 5 | 3 | 0.0008 | CRISP1, SPAM1, HYAL4 |
| | membrane | | | | |
| | Glutathione metabolism (KEGG) | 30 | 6 | 0.0016 | GCLC, GPX5, GSTA1, GSTA2, GSTA3, GSTA4 |
| | protein phosphatase inhibitor activity | 26 | 5 | 0.0046 | PPP1R10, PSMB9, PPP1R11, C13orf18, PHACTR1 |
| | ATPase activity, coupled to | 37 | 6 | 0.0047 | ABCF1, CFTR, ABCB4, TAP1, TAP2, ABCC10 |
| | transmembrane movement of | | | | |
| | substances | | | | |
| | nucleoside-triphosphatase activity | 119 | 12 | 0.0054 | ABCF1, CFTR, MCM3, PEX6, ABCB4, PSMC2, |
| | | | | | RFC3, TAP1, TAP2, WRNIP1, KATNAL1, ABCC10 |
| | tumor necrosis factor receptor | 18 | 4 | 0.0065 | LTA, LTB, TNF, TNFSF11 |
| | binding | | | | |
| | G1_to_S_cell_cycle_Reactome | 65 | 8 | 0.0067 | CCND3, CDKN1A, CREBL1, E2F3, MCM3, ORC5L, |
| | (GenMAPP) | | | | RB1, CCNA1 |
| | sulfate transport | 10 | 3 | 0.0077 | SLC26A3, SLC26A4, SLC13A1 |
| | large ribosomal subunit | 10 | 3 | 0.0077 | WASL, RPL7L1, LOC441150 |
| | glutathione transferase activity | 19 | 4 | 0.0080 | GSTA1, GSTA2, GSTA3, GSTA4 |
| Stage | Starch and sucrose metabolism | 33 | 5 | 9.96E−07 | AGL, AMY1A, AMY1B, AMY2A, RNPC3 |
| | (KEGG) | | | | |
| | alpha-amylase activity | 6 | 3 | 3.27E−06 | AMY1A, AMY1B, AMY2A |
| | hydrolase activity, acting on glycosyl | 61 | 5 | 2.21E−05 | AGL, AMY1A, AMY1B, AMY2A, RNPC3 |
| | bonds | | | | |
| | chloride channel activity | 17 | 3 | 0.0001 | CLCA1, CLCA2, CLCA4 |
| | voltage-gated chloride channel | 18 | 3 | 0.0001 | CLCA1, CLCA2, CLCA4 |
| | activity | | | | |
| | GTPase activity | 145 | 6 | 0.0002 | GBP1, GBP2, GBP3, GBP4, GBP5, GBP6 |
| | Digestion | 53 | 4 | 0.0002 | AMY1A, AMY1B, AMY2A, RNPC3 |
| | lysosphingolipid and | 7 | 2 | 0.0006 | EDG1, EDG7 |
| | lysophosphatidic acid receptor | | | | |
| | activity | | | | |
| | chloride transport | 42 | 3 | 0.0016 | CLCA1, CLCA2, CLCA4 |
| Size | centrosome cycle | 5 | 1 | 0.0090 | NPM1 |
| | cell aging | 5 | 1 | 0.0090 | NPM1 |
| | coenzyme A biosynthesis | 5 | 1 | 0.0090 | PANK3 |
| Microvascular | carbonate dehydratase activity | 16 | 5 | 1.13E−05 | CA1, CA2, CA3, CA4, CA8 |
| invasion | Nitrogen metabolism (KEGG) | 18 | 5 | 2.14E−05 | CA1, CA2, CA3, CA4, CA8 |
| | Phenylalanine metabolism (KEGG) | 5 | 3 | 7.69E−05 | LPO, MPO, EPX |
| | Stilbene, coumarine and lignin | 7 | 3 | 0.0003 | LPO, MPO, EPX |
| | biosynthesis (KEGG) | | | | |
| | one-carbon compound metabolism | 30 | 5 | 0.0003 | CA1, CA2, CA3, CA4, CA8 |
| | Methane metabolism (KEGG) | 8 | 3 | 0.0004 | LPO, MPO, EPX |
| | protein serine/threonine phosphatase | 10 | 3 | 0.0009 | PPM1D, PPM1E, PPM2C |
| | complex | | | | |
| | double-strand break repair | 11 | 3 | 0.0012 | NBN, PRKDC, RAD21 |
| | peroxidase activity | 25 | 4 | 0.0014 | LPO, MPO, EPX, PXDNL |
| | ribonuclease P activity | 6 | 2 | 0.0057 | POP4, POP1 |
| | response to oxidative stress | 37 | 4 | 0.0062 | LPO, MPO, EPX, OXR1 |
| | Oxidative phosphorylation (KEGG) | 60 | 5 | 0.0069 | ATP6V1C1, COX6C, UQCRB, UQCRFS1, ATP6V1H |
| | opioid receptor activity | 7 | 2 | 0.0078 | NPBWR1, OPRK1 |
| | potassium channel activity | 90 | 6 | 0.0093 | KCNQ3, KCNS2, KCNB2, KCNV1, KCNK9, CNGB3 |
| Portal vein | hydrolase activity, acting on carbon- | 5 | 3 | 1.52E−05 | VNN2, VNN1, VNN3 |
| invasion | nitrogen | | | | |
| | MHC class I protein complex | 16 | 4 | 2.88E−05 | ULBP3, ULBP2, ULBP1, RAET1E |
| | hydrolase activity, acting on carbon- | 7 | 3 | 5.21E−05 | VNN2, VNN1, VNN3 |
| | nitrogen (but not peptide) bonds | | | | |
| | antigen presentation | 19 | 4 | 5.96E−05 | ULBP3, ULBP2, ULBP1, RAET1E |
| | MHC class I receptor activity | 21 | 4 | 9.04E−05 | ULBP3, ULBP2, ULBP1, RAET1E |
| | nitrogen compound metabolism | 12 | 3 | 0.0003 | VNN2, VNN1, VNN3 |
| | ion transporter activity | 15 | 3 | 0.0006 | SLC22A1, SLC22A3, SLC22A2 |
| | fibrinolysis | 7 | 2 | 0.0027 | LPA, PLG |
| | MAP kinase kinase kinase activity | 8 | 2 | 0.0036 | MAP3K4, MAP3K5 |
| | protein kinase A binding | 8 | 2 | 0.0036 | AKAP7, AKAP12 |
| | Riboflavin metabolism (KEGG) | 9 | 2 | 0.0046 | ENPP1, ENPP3 |
| | protein localization | 10 | 2 | 0.0057 | AKAP7, SNX9 |
| | Pantothenate and CoA biosynthesis | 10 | 2 | 0.0057 | ENPP1, ENPP3 |
| | (KEGG) | | | | |
| | Nicotinate and nicotinamide | 11 | 2 | 0.0069 | ENPP1, ENPP3 |
| | metabolism (KEGG) | | | | |
| | natural killer cell activation | 11 | 2 | 0.0069 | ULBP3, ULBP2 |
| | spindle pole | 12 | 2 | 0.0082 | LATS1, KATNA1 |
| Encapsulation | response to drug | 15 | 2 | 8.02E−05 | ABCB4, SEMA3C |
| | blood coagulation | 70 | 2 | 0.0018 | CD36, HGF |
| | fatty acid metabolism | 70 | 2 | 0.0018 | CD36, CROT |
| | transmembrane receptor protein | 72 | 2 | 0.0019 | EGFR, SEMA3C |
| | tyrosine kinase signaling pathway | | | | |
| | epidermal growth factor receptor | 6 | 1 | 0.0054 | EGFR |
| | activity | | | | |

### High-Level Copy Number Changes

In total, 33 amplifications and 10 homozygous deletions (HD) were identified (see Table 8). Most high-level copy number changes were observed in a single case, but some of them appeared recurrently. For example, amplification on 8q11.1-8q24.3 containing MYC and EIF3S3 was observed in 10 cases and amplification on 11q13.2-11q13.3 containing CCND1, FGF4, FGF3 and ORAOV1 in 5 cases. In addition, amplifications on 1q31.1-1q43, 1q43-1q44, 13q31.1-13q34, and 17q12-17q25.3 were detected in 4 cases. All HDs were observed in a single case except for 9p21.3 containing CDKN2A and CDKN2B tumor suppressor.

TABLE 8
High-level copy number changes in 76 HCCs.

| | | | | | | Number of | |
|---|---|---|---|---|---|---|---|
| | Probe | Chromosome | Map position^{a} | RAR^{b} | Cytoband | cases | Cancer-related genes |
| Amp1 | RP4-706A17- | 1 | 143352667-144380209 | G1 | 1q21.1 | 1 | PDZK1 |
| | RP11-533N14 | | | | | | |
| Amp2 | RP11-422P24- | 1 | 150704923-151839277 | G1 | 1q22 | 1 | TPM3, ADAR, RPS27, HAX1, PYGO2, |
| | RP11-307C12 | | | | | | CKS1B, ADAM15 |
| Amp3 | RP11-98F1- | 1 | 152090103-152897176 | G1 | 1q22 | 1 | HDGF, CCT3, PRCC, RIT1, ETV3 |
| | RP11-172I6 | | | | | | |
| Amp 4 | RP11-190A12 | 1 | 156557891-156696755 | G1 | 1q23.2 | 1 | CRP |
| Amp 5 | RP11-430G6- | 1 | 159688698-179511167 | G1 | 1q23.3-1q25.3 | 2 | SELE, SELL, SELP, LAMC2 |
| | RP11-71D4 | | | | | | |
| Amp 6 | RP11- | 1 | 183908427-185875252 | G1 | 1q31.1 | 1 | TPR, PTGS2 |
| | 108M21- | | | | | | |
| | RP11-336D15 | | | | | | |
| Amp 7 | RP11-445K1- | 1 | 186717333-236040328 | G2 | 1q31.1-1q43 | 4 | KIF14, ELF3, TGFB2 |
| | RP11-359A17 | | | | | | |
| Amp 8 | RP11-80B9- | 1 | 237216929-244440465 | G2 | 1q43-1q44 | 4 | AKT3 |
| | RP11-438H8 | | | | | | |
| Amp 9 | RP11- | 3 | 8869819-9236203 | | 3p25.3 | 1 | CAV3, OXTR |
| | 105K13- | | | | | | |
| | RP11-334L22 | | | | | | |
| Amp 10 | RP11-89F18- | 3 | 23404803-24311473 | | 3p24.3 | 1 | |
| | RP11-18L17 | | | | | | |
| Amp 11 | RP11- | 3 | 35590143-36744654 | | 3p22.3 | 1 | |
| | 380G10- | | | | | | |
| | RP11-134C18 | | | | | | |
| Amp 12 | RP11- | 3 | 52556261-52839811 | | 3p21.1 | 1 | |
| | 447A21- | | | | | | |
| | RP5-966M1 | | | | | | |
| Amp 13 | RP11-154D3- | 3 | 62016735-62347194 | | 3p14.2 | 1 | |
| | RP11-204J18 | | | | | | |
| Amp 14 | RP11- | 3 | 173181079-173855790 | | 3q26.2-3q26.31 | 2 | SKIL, PLD1, ECT2 |
| | 362K14- | | | | | | |
| | RP11-44A1 | | | | | | |
| Amp 15 | RP11-494P23- | 5 | 67322896-67677101 | | 5q13.1 | 1 | PIK3R1, CCNB |
| | RP11-421A17 | | | | | | |
| Amp 16 | RP11-175A4- | 6 | 33467577-34013145 | G5 | 6p21.32 | 1 | HMGA1 |
| | RP3-468B3 | | | | | | |
| Amp 17 | RP3-431A14- | 6 | 36751256-56905292 | G5 | 6p21.31-6p12.1 | 2 | PIM1, TFEB, CCND3, VEGF |
| | RP11-472M19 | | | | | | |
| Amp 18 | RP11- | 6 | 134784496-135663804 | | 6q23.2 | 1 | MYB |
| | 557H15- | | | | | | |
| | RP1-32B1 | | | | | | |
| Amp 19 | RP11-44M6 | 7 | 99680294-99845417 | | 7q22.1 | 1 | EPO |
| Amp 20 | RP5-905M6 | 7 | 110673548-110781794 | G8 | 7q31.1 | 1 | |
| Amp 21 | RP11-350F16- | 8 | 47816604-145778719 | G9 | 8q11.1-8q24.3 | 10 | PRKDC, MCM4, SNAI2, LYN, MOS, |
| | RP11-349C2 | | | | | | PLAG1, COPS5, TPD52, E2F5, MMP16, |
| | | | | | | | NBS1, EIF3S3, MYC, KCNK9, |
| | | | | | | | PTK2, EIF2C2 |
| Amp 22 | RP11-563H8- | 9 | 71314535-76253531 | | 9q21.13 | 1 | ANXA1 |
| | RP11-422N19 | | | | | | |
| Amp 23 | RP11- | 10 | 74746892-76882563 | | 10q22.2 | 1 | ANXA7, PLAU, VDAC2 |
| | 345K20- | | | | | | |
| | RP11-399K21 | | | | | | |
| Amp 24 | RP1-85M6- | 11 | 32978034-33595628 | | 11p13 | 1 | |
| | RP1-316D7 | | | | | | |
| Amp 25 | RP11-569N5- | 11 | 68186960-69323966 | | 11q13.2-11q13.3 | 5 | ORAOV1, FGF4, FGF3, CCND1 |
| | RP11-300I6 | | | | | | |
| Amp 26 | RP11- | 11 | 91893710-93189097 | | 11q21 | 1 | |
| | 533H15- | | | | | | |
| | RP11-236L3 | | | | | | |
| Amp 27 | RP11-437F6- | 12 | 23609734-25447853 | | 12p12.1 | 1 | KRAS2 |
| | RP11-707G18 | | | | | | |
| Amp 28 | RP11- | 13 | 84781263-112901415 | G11 | 13q31.1-13q34 | 4 | FGF14, TFDP1, CUL4A, GAS6, CDC16 |
| | 376H15- | | | | | | |
| | RP11-265C7 | | | | | | |
| Amp 29 | RP11-390P24- | 17 | 34803850-78374826 | G12 | 17q12-17q25.3 | 4 | ERBB2, GRB7, CSF3, TOP2A, |
| | RP11-567O16 | | | | | | CCR7, BRCA1, ETV4, GRN, |
| | | | | | | | COL1A1, CACNA1G, HLF, MPO, |
| | | | | | | | TBX2, RAC3, AXIN2, PRKCA, SOX9, |
| | | | | | | | GRB2, TIMP2, RAC3 |
| Amp 30 | CTD-3149D2- | 19 | 17561412-22729110 | | 19p13.11-19p12 | 2 | JUND, JAK3, EDG4 |
| | CTC-451A6 | | | | | | |
| Amp 31 | CTD-2057D4- | 19 | 34956729-36979856 | G13 | 19q12 | 2 | CCNE1 |
| | CTC-416D1 | | | | | | |
| Amp 32 | RP4-633O20- | 20 | 34762707-61041280 | G14 | 20q11.23-20q13.33 | 1 | SRC, TOP1, MYBL2, MMP9, NCOA3, |
| | RP4-563E14 | | | | | | PTPN1, ZNF217, STK6, BMP7 |
| Amp 33 | CTA-390B3- | 22 | 36114798-42644660 | | 22q13.1-22q13.2 | 1 | PDGFB, EP300, BIK |
| | RP3-388M5 | | | | | | |
| HD1 | RP5-1043L3- | 1 | 87387994-88674564 | L4 | 1p22.2 | 1 | GTF2B |
| | RP11-427B20 | | | | | | |
| HD2 | RP11-351J23- | 6 | 167866346-168197568 | L7 | 6q27 | 1 | UNC93A, MLLT4 |
| | RP3-470B24 | | | | | | |
| HD3 | RP11- | 8 | 2898583-4066807 | L8 | 8p23.2 | 1 | CSMD1 |
| | 336N16- | | | | | | |
| | RP11-45M12 | | | | | | |
| HD4 | RP5-991O23- | 8 | 5326019-8677720 | L8 | 8p23.2 | 1 | DEFB1 |
| | RP11-211C9 | | | | | | |
| HD5 | RP11-809L8- | 8 | 18249257-18644382 | L8 | 8p22 | 1 | NAT1, NAT2, PSD3 |
| | RP11-161I2 | | | | | | |
| HD6 | RP11- | 9 | 20996400-25069411 | L9 | 9p21.3 | 2 | CDKN2B, CDKN2A |
| | 113D19- | | | | | | |
| | RP11-468C2 | | | | | | |
| HD7 | RP11-59H1- | 12 | 12770043-13592296 | | 12p13.1 | 1 | CDKN1B |
| | RP11-4N23 | | | | | | |
| HD8 | RP11-174I10 | 13 | 47897821-47960646 | L13 | 13q14.2 | 1 | RB1 |
| HD9 | RP11-327P2 | 13 | 51243101-51412205 | L13 | 13q14.3 | 1 | DDX26 |
| HD10 | RP11-424E21- | 13 | 65400726-66421705 | | 13q21.32 | 1 | |
| | RP11-10M21 | | | | | | |

^{a}The mapping position refers to the UCSC genome browser (http://genome.ucsc.edu/; May 2004 freeze)
^{b}RARs overlapping with high copy number changes
Amp, amplification;
HD, homozygous deletion

### Putative Hepatocarcinogenesis Related Oncogenes in RARs on 1q

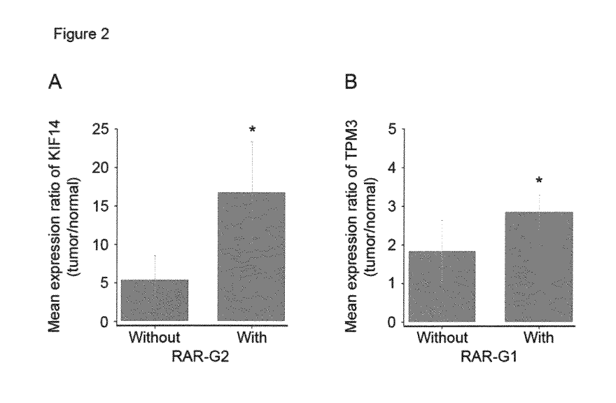

Many of the high-level copy number changes were found within RARs. Sixteen out of 33 amplifications and 8 out of 10 HDs overlapped with RAR-Gs and -Ls, respectively (see Table 8). We assumed overlapped alterations within RARs might be more closely related to carcinogenesis. Thus, we examined the RNA profile of the putative cancer related genes as follows in RAR-G1 and -G2, which most extensively overlapped with amplifications; TPM3, RPS27, HAX1, PYGO2, CKS1B, ADAM15, CCT3, PRCC, KIF14, ELF3, TGFB2, and AKT3. Total RNA was available for 20 cases out of 76 HCCs, of which 13 cases are RAR-G1 positive, and 12 cases are RAR-G2 positive. RNA expression levels of most candidate genes in the HCCs are higher than control RNA levels measured using normal liver tissue from each patient. Especially, KIF14 and TPM3 are highly expressed in HCC and their expression is significantly correlated with copy number status (FIGS. 2A and 2B). Mean intensity ratio (tumor/normal) of KIF14 in RAR-G2 positive cases was 16.8 (95% CI: 10.14-23.38), while 5.3 in RAR-G2 negative cases (95% CI: 2.14-8.54). Mean intensity ratio of TPM3 was significantly higher in RAR-G1 positive cases (2.84, 95% CI: 2.39-3.28) than RAR-G1 negative group (1.83, 95% CI: 1.02-2.64). CKS1B also showed higher expression in RAR-G1 positive group than negatives (1.42 versus 2.21), but the difference is not statistically significant.

Association with Survival

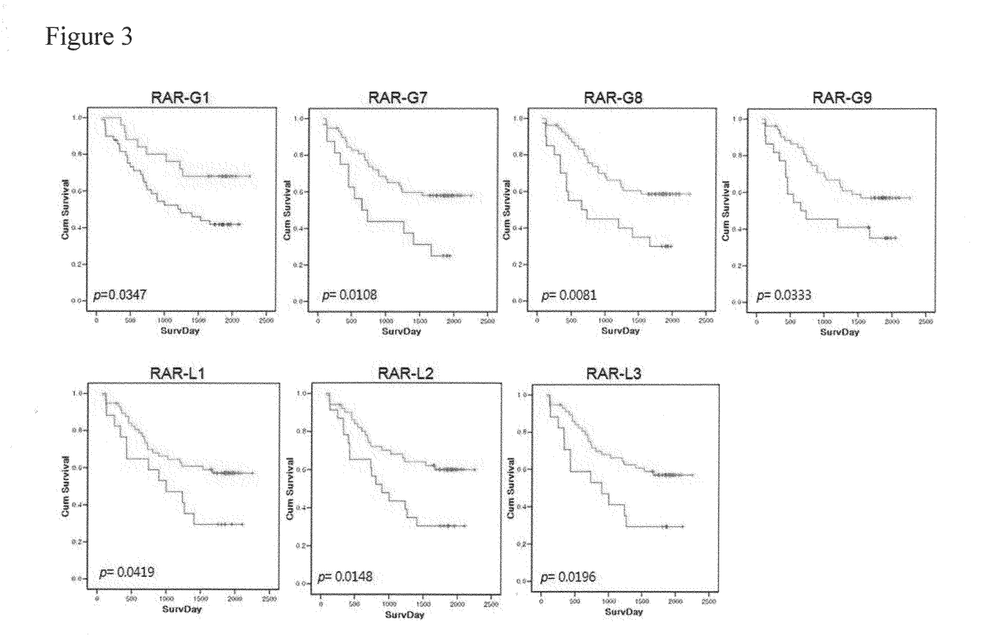

Survival analysis was performed to assess the prognostic values of the MARs. In univariate analysis, MAR-G1 (p=0.0347), MAR-G7 (p=0.0108), MAR-G8 (p=0.0081), MAR-G9 (p=0.0333), MAR-L1 (p=0.0419), MAR-L2 (p=0.0148), MAR-L3 (p=0.0196) were significantly associated with poor survival (FIG. 3). When we compared the high MAR-G group (more than 5 MAR-Gs per case, n=27) and low MAR-G group (less than 3 MAR-Gs per single case, n=33), high MAR-G group showed significantly lower survival than low MAR-G group (p=0.0092). There was no difference between high and low MAR-L groups.

The present invention is a diagnosis method for determining prognosis of liver cancer by using comparative genomic hybridization. With the present invention, it is possible to get early diagnosis and prognosis of liver cancer. The present invention is simple to use for diagnosis or prognosis of liver cancer because it is specially designed for liver cancer with low density, thus useful for general hospital to test liver cancer. With the method of PCR using one species of DNA test marker, it is limited to get correct diagnosis or prognosis. Therefore it is ideal due to simplicity and efficiency when DNA analysis is simultaneously carried out through whole genome by using core makers which are capable of diagnosis or prognosis of liver cancer significantly. The technique of microarray is proper to simultaneously analyze DNA markers of prognosis or diagnosis of liver cancer. Array CGH is suitable for this purpose. The present invention provides markers which are most significantly correlated to prognosis or diagnosis of the liver cancer and provides test kits for liver cancer by using the markers. If this present invention is selected as an index of liver cancer test, it is expected that more than 10,000 tests would be done in a year.

### REFERENCES

1. Blum H E. Hepatocellular carcinoma: therapy and prevention. World J Gastroenterol 2005;11:7391-400.
2. El-Serag H B. Hepatocellular carcinoma: recent trends in the United States. Gastroenterology 2004;127:S27-S34.
3. Llovet J M, Burroughs A, Bruix J. Hepatocellular carcinoma. Lancet 2003;362:1907-17.
4. Vissers L E, Veltman J A, van Kessel A G, Brunner H G. Identification of disease genes by whole genome CGH arrays. Hum Mol Genet 2005;14:R215-R223.
5. Pinkel D, Albertson D G. Array comparative genomic hybridization and its applications in cancer. Nat Genet 2005;37:S11-S17.
6. Hashimoto K, Mori N, Tamesa T, Okada T, Kawauchi S, Oga A, Furuya T, Tangoku A, Oka M, Sasaki K. Analysis of DNA copy number aberrations in hepatitis C virus-associated hepatocellular carcinomas by conventional CGH and array CGH. Mod Pathol 2004;17:617-22.
7. Moinzadeh P, Breuhahn K, Stützer H, Schirmacher P. Chromosome alterations in human hepatocellular carcinomas correlate with aetiology and histological grade—results of an explorative CGH meta-analysis. Br J Cancer 2005;92:935-41.
8. Zimonjic D B, Keck C L, Thorgeirsson S S, Popescu N C. Novel recurrent genetic imbalances in human hepatocellular carcinoma cell lines identified by comparative genomic hybridization. Hepatology 1999;29:1208-14.
9. Pinkel D, Segraves R, Sudar D, Clark S, Poole I, Kowbel D, Collins C, Kuo W L, Chen C, Zhai Y, Dairkee S H, Ljung B M, et al. High resolution analysis of DNA copy number variation using comparative genomic hybridization to microarrays. Nat Genet 1998;20:207-11.
10. Patil M A, Gütgemann I, Zhang J, Ho C, Cheung S T, Ginzinger D, Li R, Dykema K J, So S, Fan S T, Kakar S, Furge K A, et al. Array-based comparative genomic hybridization reveals recurrent chromosomal aberrations and Jab1 as a potential target for 8q gain in hepatocellular carcinoma. Carcinogenesis 2005;26:2050-7.
11. Kim T M, Yim S H, Lee J S. Kwon M S, Ryu J W, Kang H M, Fiegler H, Carter N P, Chung Y J. Genome-wide screening of genomic alterations and their clinicopathologic implications in non-small cell lung cancers. Clin Cancer Res 2005;11:8235-42.
12. Kim M Y, Yim S H, Kwon M S, Kim T M, Shin S H, Kang H M, Lee C, Chung Y J. Recurrent genomic alterations with impact on survival in colorectal cancer identified by genome-wide array comparative genomic hybridization. Gastroenterology 2006; 131:1913-24.
13. Fiegler H, Carr P, Douglas E J, Burford D C, Hunt S, Scott C E, Smith J, Vetrie D, Gorman P, Tomlinson I P, Carter N P. DNA microarrays for comparative genomic hybridization based on DOP-PCR amplification of BAC and PAC clones. Genes Chromosomes Cancer 2003;36:361-74.
14. Kim S Y, Nam S W, Lee S H, Park W S, Yoo N J, Lee J Y, Chung Y J. ArrayCyGHt: a web application for analysis and visualization of array-CGH data. Bioinformatics 2005;21:2554-5.
15. Livak K J, Schmittgen T D. Analysis of relative gene expression data using real-time quantitative PCR and the 2(-Delta Delta C(T)) Method. Methods 2001;25:402-8.
16. Cheng J, Sun S, Tracy A, Hubbell E, Morris J, Valmeekam V, Kimbrough A, Cline M S, Liu G, Shigeta R, Kulp D, Siani-Rose M A. NetAffx Gene Ontology Mining Tool: a visual approach for microarray data analysis. Bioinformatics 2004;20:1462-3.
17. Dahlquist K D, Salomonis N, Vranizan K, Lawlor S C, Conklin B R. GenMAPP, a new tool for viewing and analyzing microarray data on biological pathways. Nat Genet 2002;31:19-20.
18. Lomax J, Ashburner M, Foulger R, Eilbeck K, Lewis S, Marshall B, Mungall C, Richter J, Rubin G M, Blake J A, Bult C, Dolan M, et al. The Gene Ontology (GO) database and informatics resource. Nucleic Acids Res 2004;32:D258-D261.
19. Kanehisa M, Goto S, Kawashima S, Nakaya A. The KEGG databases at GenomeNet. Nucleic Acids Res 2002;30:42-6.
20. Kim T M, Jung Y C, Rhyu M G, Jung M H, Chung Y J. GEAR: genomic enrichment analysis of regional DNA copy number changes. Bioinformatics, 2007; doi: 10.1093/bioinformatics/btm582.
21. Yasui K, Arii S, Zhao C, Imoto I, Ueda M, Nagai H, Emi M, Inazawa J. TFDP1, CUL4A, and CDC16 identified as targets for amplification at 13q34 in hepatocellular carcinomas. Hepatology 2002;35:1476-84.
22. Fang W, Piao Z, Simon D, Sheu J C, Huang S. Mapping of a minimal deleted region in human hepatocellular carcinoma to 1p36.13-p36.23 and mutational analysis of the RIZ (PRDM2) gene localized to the region. Genes Chromosomes Cancer 2000;28:269-75.
23. Guan X Y, Sham J S, Tai L S, Fang Y, Li H, Liang Q. Evidence for another tumor suppressor gene at 17p13.3 distal to TP53 in hepatocellular carcinoma. Cancer Genet. Cytogenet 2003;140:45-8.
24. Spangenberg H C, Thimme R, Blum H E. Serum markers of hepatocellular carcinoma. Semin Liver Dis 2006;26:385-90.
25. Ribatti D, Marzullo A, Gentile A, Longo V, Nico B, Vacca A, Dammacco F. Erythropoietin/erythropoietin-receptor system is involved in angiogenesis in human hepatocellular carcinoma. Histopathology 2007;50:591-6.
26. Okamoto H, Yasui K, Zhao C, Arii S, Inazawa J. PTK2 and EIF3S3 genes may be amplification targets at 8q23-q24 and are associated with large hepatocellular carcinomas. Hepatology 2003;38:1242-9.
27. Reeves M E, DeMatteo R P. Genes and viruses in hepatobiliary neoplasia. Semin Surg Oncol 2000;19:84-93.
28. Corson T W, Huang A, Tsao M S, Gallie B L. KIF14 is a candidate oncogene in the 1q minimal region of genomic gain in multiple cancers. Oncogene 2005;24:4741-53.
29. Lamant L, Dastugue N, Pulford K, Delsol G, Mariame B. A new fusion gene TPM3-ALK in anaplastic large cell lymphoma created by a (1 ;2)(q25;p23) translocation. Blood 1999;93:3088-95.
30. Jou Y S, Lee C S, Chang Y H, Hsiao C F, Chen C F, Chao C C, Wu L S, Yeh S H, Chen D S, Chen P J. Clustering of minimal deleted regions reveals distinct genetic pathways of human hepatocellular carcinoma. Cancer Res 2004;64:3030-6.
31. Wang Y, Wu M C, Sham J S, Zhang W, Wu W Q, Guan X Y. Prognostic significance of c-myc and AIB1 amplification in hepatocellular carcinoma. A broad survey using high-throughput tissue microarray. Cancer 2002;95:2346-52.
32. Herzer K, Sprinzl M F, Galle P R. Hepatitis viruses: live and let die. Liver Int 2007;27: 293-301.
33. Radaeva S, Jaruga B, Hong F; Kim W H, Fan S, Cai H, Strom S, Liu Y, El-Assal O, Gao B. Interferon-alpha activates multiple STAT signals and down-regulates c-Met in primary human hepatocytes. Gastroenterology 2002;122:1020-34.
34. Greenbaum L E. Cell cycle regulation and hepatocarcinogenesis. Cancer Biol Ther 2004;3:1200-7.
35. Herman J G, Meadows G G. Increased class 3 semaphorin expression modulates the invasive and adhesive properties of prostate cancer cells. Int J Oncol 2007;30:1231-8.

What is claimed is:

1. A diagnostic method for determining the prognosis of hepatocellular carcinoma (HCC) comprising the steps of; (a) observing recurrently altered genomic region on a chromosome; (b) measuring variation of one or more of RAR expression variations selected from the RAR variation group consists of gains of RAR-G1 to RAR-G14 and losses of RAR-L1 to RAR-L18 as defined table 1.

2. The method of claim 1, wherein said RAR variation group consists of RAR-G1, RAR-G2, RAR-L17, RAR-G9, RAR-L5 and RAR-L8.

3. The method of claim 1, wherein the method is a method for determining the tumor stage of hepatocellular carcinoma by using RAR-L2 and RAR-L4.

4. The method of claim 1, wherein the method is a method for determining prognosis of microvascular invasion of hepatocellular carcinoma by using RAR-G9, RAR-G12, RAR-G13 and RAR-L3.

5. The method of claim 1, wherein the method is a diagnostic method for determining the portal vein invasion of the hepatocellular carcinoma by using RAR-G13, RAR-L7 and RAR-L12.

6. A diagnostic kit for determining the prognosis of hepatocellular carcinoma (HCC) comprising; (a) a microarray comprising a probe for measuring variation of one or more of RAR expression variations selected from the RAR variation group consists of gains of RAR-G1 to RAR-G14 and losses of RAR-L1 to RAR-L18 as defined table 1 for observing recurrently altered genomic region (RAR) on a chromosome; and (b) an image analysis device for measuring variation of specific genes expression on the RAR.

7. The kit of claim 6, wherein the the probe for measuring is RAR-G1, RAR-G2, RAR-L17, RAR-G9, RAR-L5 and RAR-L8.

8. A diagnostic method for determining the prognosis of hepatocellular carcinoma (HCC) comprising the step of measuring variation of one or more of gene expression variations selected from the group consists of tropomyosin 3 (TPM3), ribosomal protein S27 (RPS27), hematopoietic cell-specific Lyn substrate 1-associated protein X-1(HAX1), pygopus homolog 2 (PYGO2), CDC28 protein kinase regulatory subunit 1 (BCKS1B), a disintegrin and metalloprotease 15 (ADAM15), chaperonin subunit 3 (CCT3), papillary renal cell carcinoma (PRCC), kinesin family member 14 (KIF14), Eukaryotic initiation factor 3 (ELF3), transforming Growth Factor beta 2 (TGFB2) and protein kinase B gamma (AKT3).
